(19) United States
(12) Patent Application Publication
(10) Pub. No.: US 2010/0204263 A1
(43) Pub. Date: Aug. 12, 2010

(54) METHOD OF TREATING MULTIDRUG RESISTANT CANCERS

(76) Inventors: Alfred M. Ajami, Brookline, MA (US); Robert L. Capizzi, Philadelphia, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/522,463

(22) PCT Filed: Jan. 9, 2008
(86) PCT No.: PCT/US2008/000321

§ 371 Date: Feb. 22, 2010

Related U.S. Application Data
(60) Provisional application No. 60/879,487, filed on Jan. 9, 2007.

(51) Int. Cl. A61K 31/473; A61P 35/02

(52) U.S. Cl. 514/296

(57) ABSTRACT

A method of treating multidrug resistant cancers in a patient, comprising administering to said patient a therapeutically effective amount of a compound of formula (I) or a pharmaceutically acceptable salt thereof. The values and preferred values for variables R1 and R2 are defined herein.

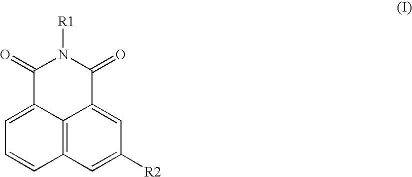

### RELATED APPLICATION

This application claims the benefit of U.S. Provisional Application No. 60/879,487 filed on Jan. 9, 2007. The entire teachings of the above application(s) are incorporated herein by reference.

### BACKGROUND OF THE INVENTION

Chemotherapeutics are commonly use for treating metastatic tumors. However, the ability of cancer cells to become simultaneously resistant to different drugs, a trait known as multidrug-resistance, remains a significant impediment to successful chemotherapy.

### SUMMARY OF THE INVENTION

In one embodiment, the present invention is a method of treating a multidrug resistant cancer in a patient. The method comprises administering to said patient a therapeutically effective amount of a compound of formula (I) or a pharmaceutically acceptable salt thereof:

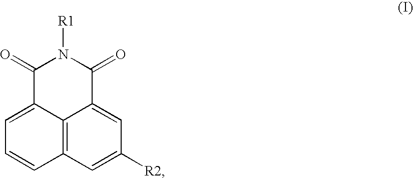

In formula (I):

R1 is —(CH₂)ₙNR3R4;

R2 is —OR5, halogen, —NR6R7, sulphonic acid, nitro, —NR5COOR5, —NR5COR5 or —OCOR5;

R3 and R4 are independently H, C1-C4 alkyl group or, taken together with the nitrogen atom to which they are bonded, a non-aromatic nitrogen-containing heterocyclic group;

each R5 is independently H or a C1-C4 alkyl group;

R6 and R7 are independently H, a C1-C4 alkyl group or, taken together with the nitrogen atom to which they are bonded, a non-aromatic nitrogen-containing heterocyclic group; and

n is an integer from 0-3.

A compound of formula (I) may be protonated with a pharmaceutically acceptable acid at R1, or, when R2 is —NR6R7, R1, R2 or both.

In another embodiment, the present invention is a method of treating refractory leukemia in a patient, comprising administering to said patient a therapeutically effective amount of a compound, of formula (I) or a pharmaceutically acceptable salt thereof.

### BRIEF DESCRIPTION OF THE DRAWINGS

FIG. 1 is a plot showing the effect of increasing concentrations of amonafide (Xanafide) on cell proliferation (MTS) assays in cell lines K562 (human leukemia). The cell lines used were either non-resistant (diamonds) or daunorubicin-resistant (circles) lines.

FIG. 2 is a plot showing the effect of daunorubicin on cell proliferation (MTS) assays in cell lines K562 (human leukemia). The cell lines used were either non-resistant (diamonds) or daunorubicin-resistant (circles) lines.

FIG. 3 is a plot showing the effect of doxorubicin on cell proliferation (MTS) assays in cell lines K562 (human leukemia). The cell lines used were either non-resistant (diamonds) or daunorubicin-resistant (circles) lines.

FIG. 4 is a plot showing the effect of idarubicin on cell proliferation (MTS) assays in cell lines K562 (human leukemia). The cell lines used were either non-resistant (diamonds) or daunorubicin-resistant (circles) lines.

FIG. 5 is a plot showing the effect of mitoxantrone on cell proliferation (MTS) assays in cell lines K562 (human leukemia). The cell lines used were either non-resistant (diamonds) or daunorubicin-resistant (circles) lines.

FIG. 6 is a plot showing the effect of etoposide on cell proliferation (MTS) assays in cell lines K562 (human leukemia). The cell lines used were either non-resistant (diamonds) or daunorubicin-resistant (circles) lines.

FIG. 7 is a plot showing the effect of amonafide (Xanafide) on cell proliferation (MTS) assays in cell lines P388 (murine leukemia) cell lines. The cell lines used were either non-resistant (diamonds) or daunorubicin-resistant (circles) lines.

FIG. 8 is a plot showing the effect of daunorubicin on cell proliferation (MTS) assays in cell lines P388 (murine leukemia) cell lines. The cell lines used were either non-resistant (diamonds) or daunorubicin-resistant (circles) lines.

FIG. 9 is a plot showing the effect of amonafide (Xanafide) on cell proliferation (clonogenic) assays in cell lines MCF7 (human breast cancer) cell lines. The cell lines used were either non-resistant (diamonds) or daunorubicin-resistant (circles) lines.

FIG. 10 is a plot showing the effect of daunorubicin on cell proliferation (clonogenic) assays in cell lines MCF7 (human breast cancer) cell lines. The cell lines used were either non-resistant (diamonds) or daunorubicin-resistant (circles) lines.

FIG. 11 is a plot showing the effect of doxorubicin on cell proliferation (clonogenic) assays in cell lines MCF7 (human breast cancer) cell lines. The cell lines used were either non-resistant (diamonds) or daunorubicin-resistant (circles) lines.

FIG. 12A and FIG. 12B are plots showing the effect of amonafide (Xanafide) on cell proliferation (SRB) assays in IGROV1 (human ovarian) cell lines (A) or IGROV1-T8, a cell line selected for resistance to topotecan (B).

FIG. 13A and FIG. 13B are plots showing the effect of amonafide (Xanafide) (A) or Daunorubicin (B) on cell proliferation (WST-1) assays in HL60NCR cells, a human promyelocytic leukemia cell line selected for resistance to vincristine in the presence or absence of PSC388, a PGP inhibitor.

FIG. 14A and FIG. 14B are plots showing the effect of amonafide (Xanafide) (A) or Daunorubicin (B) on cell proliferation (WST-1) assays in HL60/ADR cells, a human promyelocytic leukemia cell line selected for resistance to adriamycin in the presence or absence of MK571, a MRP-1 inhibitor.

FIG. 15A and FIG. 15B are plots showing the effect of amonafide (Xanafide) (A) or Daunorubicin (B) on cell proliferation (WST-1) assays in 8226/MR20 cells, a human myeloma cell line selected for resistance to mitoxantrone in the presence or absence of Fumitremorgin C (FTC), a BCRP inhibitor.

FIG. 16 is a bar plot of the Resistance Ratios of amonafide L-malate (Xanafide), daunorubicin, doxorubicin, idarubicin, mitoxantrone, etoposide and cytarabine in HL60NCR cells, a human promyelocytic leukemia cell line selected for resistance to vincristine.

FIG. 17 is a bar plot of the Resistance Ratios of amonafide L-malate (Xanafide), daunorubicin, doxorubicin, idarubicin, mitoxantrone, etoposide and cytarabine in HL60/ADR cells, a human promyelocytic leukemia cell line selected for resistance to adriamycin.

FIG. 18 is a bar plot of the Resistance Ratios of amonafide L-malate (Xanafide), daunorubicin, doxorubicin, idarubicin, mitoxantrone, etoposide and cytarabine in 8226/MR20 cells, a human myeloma cell line selected for resistance to mitoxantrone.

FIG. 19 is a bar plot of the Resistance Modifying Factors of amonafide L-malate (Xanafide), daunorubicin, doxorubicin, idarubicin, mitoxantrone, etoposide and cytarabine in HL60NCR cells, a human promyelocytic leukemia cell line selected for resistance to vincristine.

FIG. 20 is a bar plot of the Resistance Modifying Factors of amonafide L-malate (Xanafide), daunorubicin, doxorubicin, idarubicin, mitoxantrone, etoposide and cytarabine in HL60/ADR cells, a human promyelocytic leukemia cell line selected for resistance to adriamycin.

FIG. 21 is a bar plot of the Resistance Modifying Factors of amonafide L-malate (Xanafide), daunorubicin, doxorubicin, idarubicin, mitoxantrone, etoposide and cytarabine in 8226/MR20 cells, a human myeloma cell line selected for resistance to mitoxantrone.

FIG. 22A and FIG. 22B are plots of the uptake and efflux of the PGP substrate DiOC2 in K562 (human leukemia) cells (A) or K562/DOX, a cell line selected for resistance to doxorubicin (B) in the presence or absence of a Cyclosporin A (CSA), a MDR inhibitor.

FIG. 23A and FIG. 23B are plots of the uptake and efflux of amonafide in K562 (human leukemia) cells (A) or K562/DOX, a cell line selected for resistance to doxorubicin (B) in the presence or absence of a PKC412, a PGP inhibitor.

FIG. 24 is plots of the cellular accumulation of amonafide with varying concentrations of amonafide in HL60 (human promyelocytic leukemia) cells.

FIG. 25A and FIG. 25B are plots of the uptake (A) and uptake/efflux (B) of amonafide in HL60NCR cells, a human promyelocytic leukemia cell line selected for resistance to vincristine in the presence or absence of a PSC388, a PGP inhibitor.

FIG. 26A and FIG. 26B are plots of the uptake (A) and uptake/efflux (B) of amonafide in HL60/ADR cells, a human promyelocytic leukemia cell line selected for resistance to adriamycin in the presence or absence of a MK571, a MRP-1 inhibitor.

FIG. 27A and FIG. 27B are plots of the uptake (A) and uptake/efflux (B) of amonafide in 8226/MR20 cells, a human myeloma cell line selected for resistance to mitoxantrone in the presence or absence of Fumitremorgin C (FTC) a BCRP inhibitor.

FIG. 28 is a plot of the efflux of amonafide and daunorubicin in pretreatment patient cells from both patients who underwent complete remissions or those that did not.

FIG. 29 is a bar plot showing the results of permeability studies performed using Caco-2 cell monolayers. Amonafide (Xanafide) (left) was compared to daunorubicin (right) in either non-resistant (Caco-2; the left bar in each pair or bars) or daunorubicin-resistant (MDR1-MDCK; cells transfected with the human multidrug resistance gene) (the right bar in each pair of bars).

FIG. 30 is a showing the effects of Amonafide (Xanafide), PKC412 and CSA co-administration on PGP mediated digoxin efflux in either non-resistant (Caco-2; the left bar in each pair or bars) or daunorubicin-resistant (MDR1-MDCK; cells transfected with the human multi-drug resistance gene) (the right bar in each pair of bars).

FIG. 31 shows Pearson coefficients calculated for 13 drugs and 3 drug transporter genes associated with multidrug resistance ABCB1.

FIG. 32 shows Pearson coefficients calculated for 13 drugs and 3 drug transporter genes associated with multidrug resistance ABCC1.

FIG. 33 shows Pearson coefficients calculated for 13 drugs and 3 drug transporter genes associated with multidrug resistance ABCC6.

### DETAILED DESCRIPTION OF THE INVENTION

### Cellular Mechanisms of Multidrug Resistance

There are two general classes of resistance to anticancer drugs: those that impair delivery of anticancer drugs to tumor cells, and those that arise in the cancer cell itself due to genetic and epigenetic alterations that affect drug sensitivity.

Cellular mechanisms of drug resistance have been intensively studied, as experimental models can be easily generated by in vitro selection with cytotoxic agents. Cancer cells in culture can become resistant to a single drug, or a class of drugs with a similar mechanism of action, by altering the drug's cellular target or by increasing repair of drug-induced damage, frequently to DNA. After selection for resistance to a single drug, cells might also show cross-resistance to other structurally and mechanistically unrelated drugs, a phenomenon that is known as multidrug resistance.

Different types of cellular multidrug resistance have been described. Resistance to natural-product hydrophobic drugs, sometimes known as classical multidrug resistance, generally results from expression of ATP-dependent efflux pumps with broad drug specificity. These pumps belong to a family of ATP-binding cassette (ABC) transporters that share sequence and structural homology. So far, 48 human ABC genes have been identified and divided into seven distinct subfamilies (ABCA-ABCG) on the basis of their sequence homology and domain organization. Resistance results because increased drug efflux lowers intracellular drug concentrations. Drugs that are affected by classical multidrug resistance include the vinca alkaloids (vinblastine and vincristine), the anthracyclines (doxorubicin and daunorubicin), the RNA transcription inhibitor actinomycin-D and the microtubule-stabilizing drug paclitaxel.

### ATP-Binding Cassette (ABC) Transporters

An extensive review of the ABC transporter superfamily is provided in “The Human ATP-Binding Cassette (ABC) Transporter Superfamily.” Dean, Michael. Bethesda (MD): National Library of Medicine (US), NCBI; 2002 Nov. and incorporated herein by reference.

The ATP-binding cassette (ABC) transporter superfamily contains membrane proteins that translocate a wide variety of substrates across extra- and intracellular membranes, including metabolic products, lipids and sterols, and drugs. Overexpression of certain ABC transporters occurs in cancer cell lines and tumors that are multidrug resistant. Conservation of the ATP-binding domains of these genes has allowed the identification of new members of the superfamily based on nucleotide and protein sequence homology.

ABC transporters have an important role in regulating central nervous system permeability. The brain is protected against blood-borne toxins by the blood-brain barrier (BBB), and the blood-cerebrospinal-fluid (CSF) barrier. The BBB is formed by the endothelial cells of capillaries, with p-glycoprotein (PGP) located on the luminal surface, preventing the penetration of cytotoxins across the endothelium. MRP proteins such as ABCC1 are localized to the basolateral membrane of the choroid plexus, where they serve to pump the metabolic waste products of CSF into the blood. ABC transporters also seem to protect testicular tissue and the developing fetus in a similar manner. In the testis, as in the brain, PGP transports toxins into the capillary lumen. ABCC1, on the other hand, is localized on the basolateral surface of Sertoli cells, protecting sperm within the testicular tubules. In the placenta, PGP is localized on the apical syncytiotrophoblast surface, where it can protect the fetus from toxic cationic xenobiotics. MRP family members and the half-transporter ABCG2 are also localized in placenta. ABCC1 and other isoforms might be involved in protecting fetal blood from toxic organic anions and excreting glutathione/glucuronide metabolites into the maternal circulation.

Whereas ABC transporters are expressed in the brain, testis and placenta to protect these ‘sanctuaries’ from cytotoxins, the liver, gastrointestinal tract and kidney use them to excrete toxins, protecting the entire organism. PGP is localized in the apical membranes of hepatocytes, where it transports toxins into bile. In humans, MRP3 is localized to the basolateral surface of hepatocytes, where it transports organic anions from liver back into the bloodstream. A similar role might exist for MRP6, which has been found to be expressed at high levels by liver cells. MRP2 (cMOAT) is also localized on the apical surface of hepatocytes, where it transports bilirubin-glucuronide and other organic anions into bile. Mutations that disrupt MRP2 function cause bilirubin accumulation and jaundice in rats and in patients with Dubin-Johnson syndrome. Mutations in BSEP are associated with progressive familial intrahepatic cholestasis type-2, which is characterized by reduced secretion of bile salts and hepatic failure. Finally, MDR2 functions as a phosphatidylcholine trans-locase, which reduces the toxicity of bile salts. Loss of MDR2 function results in progressive familial intrahepatic cholestasis type-3.

In the gastrointestinal tract, PGP is localized in apical membranes of mucosal cells, where it extrudes toxins, forming a first line of defense. Increased tissue concentrations of PGP substrates in Mdr1a/Mdr1b-knockout mice indicate that PGP might have a significant role in determining oral drug bioavailability. Studies have shown increased tissue absorption of putative PGP substrates following oral administration when a PGP inhibitor is administered concurrently. Additionally, PGP actively secretes intravenously administered drugs into the gastrointestinal tract. In contrast to PGP, ABCC1 is located in the basolateral membrane of mucosal cells, and therefore transports substrates into the interstitium and the bloodstream, rather than across the apical surface into the intestinal lumen. Consistent with the absence of expression on the apical surface, ABCC1-null mice have not been found to have alterations in drug pharmacokinetics. MRP2, on the other hand, localizes to the canalicular membrane of hepatocytes and the apical surface of epithelial cells, and has a primary role in the excretion of bilirubin-glucuronide. Studies confirmed that MRP2 was capable of mediating drug efflux, and a recent study showed increased bioavailability of a food-derived carcinogen—2-amino-1-methyl-6-phenylimidazo[4,5-b]pyridine—in Mrp2-null rats. This indicates that MRP2, like PGP, might also regulate drug bioavailability.

### ABC Transporters in Human Cancers

Cells exposed to toxic compounds can develop resistance by a number of mechanisms including decreased uptake, increased detoxification, alteration of target proteins, or increased excretion. Several of these pathways can lead to multidrug resistance (MDR) in which the cell is resistant to several drugs in addition to the initial compound. This is a particular limitation to cancer chemotherapy, and the MDR cell often displays other properties, such as genome instability and loss of checkpoint control, that complicate further therapy. ABC genes play an important role in MDR, and at least six genes are associated with drug transport.

Three ABC genes appear to account for nearly all of the MDR tumor cells in both human and rodent cells. These are ABCB1 (PGP/MDR1), ABCC1 (MRP1), and ABCG2 (MXR/BCRP)(Table 1). No other genes have been found overexpressed in cells that display resistance to a wide variety of drugs and in cells from mice with disrupted Abcb1a, Abcb1b, and Abcc1 genes; the Abcg2 gene was overexpressed in all MDR cell lines derived from a variety of selections.

TABLE 1
ABC transporters involved in drug resistance.

| Gene | Substrates | Inhibitors |
|---|---|---|
| ABCB1 | Colchicine, doxorubicin, VP16 | Verapamil, PSC833, |
| | (etoposide), Adriamycin, vinblastine, | GG918, V-104, |
| | digoxin, saquinivir, paclitaxel | Pluronic L61 |
| ABCC1 | Doxorubicin, daunorubicin, vincristine, | Cyclosporin A, V-104 |
| | VP16, colchicines, VP16, rhodamine | |
| ABCC2 | Vinblastine, sulfinpyrazone | |
| ABCC3 | Methotrexate, VP16 | |
| ABCC4 | Nucleoside monophosphates | |
| ABCC5 | Nucleoside monophosphates | |
| ABCG2 | Mitoxantrone, topotecan, doxorubicin, | Fumitremorgin C, |
| | daunorubicin, CPT-11, rhodamine | GF120918 |

Although it seems likely that cancer cells use several different types of ABC transporter to gain drug resistance, most clinical studies have focused on ABCB1. Early studies showed that ABCB1 was highly expressed in colon, kidney, adrenocortical and hepatocellular cancers.

Expression of ABCC1 has also been analysed in clinical samples. Antibodies against ABCC1 seem to be more specific than those that recognize ABCB1, and ABCC1 is highly expressed in leukemias, esophageal carcinoma and non-small-cell lung cancers.

### Leukemia

The most uniform associations between ABCB1 (MDR1/PGP) expression and drug resistance have been reported in acute myelogenous leukemia (AML). ABCB1 expression has been reported in leukemic cells from about one-third of patients with AML at the time of diagnosis, and more than 50% of patients at relapse; higher levels occur in certain subtypes, including secondary leukemias. ABCB1 expression is correlated with a reduced complete remission rate, and a higher incidence of refractory disease. Recent studies report that ABCB1 expression is associated with a poorer prognosis. These clinical results are supported by ex vivo studies of leukemia cells, which have shown that ABCB1 expression reduces the intracellular accumulation of daunorubicin. In addition, administration of a ABCB1 inhibitor increases daunorubicin accumulation in leukemic cells.

ABCC1 expression has also been evaluated in leukemia. Increased ABCC1 expression has been reported in chronic lymphocytic and pro-lymphocytic leukemia cells. Expression levels are less frequently elevated in AML cells (10-34%) and these studies lead to different conclusions about whether ABCC1 confers a poor prognosis. So far, the largest trial in untreated patients found no correlation between ABCC1 expression and prognosis, but observed a correlation between ABCB1 expression and prognosis. Finally, low expression levels of BCRP/MXR have been observed in AML cells. Taken together, the clinical data support a role for ABCB1 in drug resistance in AML patients, and for ABCC1 expression in chronic lymphocytic and prolymphocytic leukemias.

### Breast Cancer

A 1997 meta-analysis of 31 reports from 1989-1996 found that 41% of breast tumors expressed ABCB1. ABCB1 expression increased after therapy and was associated with a greater likelihood of treatment failure. Recent imaging studies using 99 mTc (technetium)-sestamibi (Cardiolite), a transport substrate recognized by ABCB1, indicate that its activity is increased in breast carcinomas.

Whether the ABCC1 expression levels associated with breast cancer are enough to confer drug resistance is not yet resolved. As ABCC1 is expressed ubiquitously, it is not surprising that using reverse transcriptase polymerase chain reaction (RT-PCR), ABCC1 mRNA can be detected in all breast cancer samples at levels comparable to that in normal tissues. One immunohistochemical analysis of a series of resected invasive primary breast carcinomas reported a correlation between relapse-free survival and ABCC1 expression.

### Other Solid Tumors

In ovarian cancer samples, 16-47% were found to express ABCB1, as measured by immunohistochemistry. Critical analysis of these data reveals that ABCB1 is expressed by only about 20% of ovarian cancers when samples were taken at diagnosis. This makes it difficult to demonstrate a correlation between expression and outcomes, such as disease-free survival, particularly given the importance of cisplatin in therapy.

In lung cancer samples, MDR1 mRNA expression was reported to be increased in 15-50% of tumors. The incidence of ABCC1 expression is much higher (about 80%) in small-cell lung cancer (SCLC) samples. ABCC1 expression was detected in 100% of non-small-cell lung cancers (NSCLC), with higher levels noted in 30% of the samples. This might not be surprising, given its ubiquitous expression in normal lung tissue. Immunohistochemical studies confirmed the predominantly plasma-membrane localization pattern of ABCC1.

Sarcomas represent another malignancy in which ABCB1 expression seems to be important for drug resistance. Immunohistochemical studies of both soft-tissue sarcomas and osteosarcomas revealed a strong association between ABCB1 expression, relapse-free survival and overall survival. Other methodologies, however, have been used to substantiate and refute these findings, and there has been no consensus regarding the effect of ABCB1 on survival in sarcomas.

### Reversal of Drug Resistance in Cancer

Since the early 1980s, many agents have been investigated for their ability to reverse ABCB1-mediated multidrug resistance in cancer patients. Examples include verapamil, the phenothiazines, quinidine, quinacrine, quinine, amiodarone, several neuroleptics, tamoxifen, progesterone, cyclosporin A, dexverapamil, dexniguldipine, GF-902128, PSC-833 and VX-710. Agents already in use for other indications, but discovered to also inhibit ABCB1, were tested in the first clinical trials. These agents were found to be weak inhibitors that were toxic at high doses. In subsequent trials, most notably those with cyclosporin A and dexverapamil, it became clear that surrogate markers would be needed to evaluate efficacy. It has also become clear that a number of complications arise in treating cancer patients with these types of drug.

### Treatment of Multidrug Resistance Cancer

An alternative approach to the reversal of multidrug resistance is the use of drug products which are not substrates for the drug transporters responsible for the multidrug resistance phenotype.

The present invention is based on a discovery that the compounds of formula (I) and pharmaceutically acceptable salts thereof, and specifically a compound of formula (II) known as amonafide (Xanafide) and pharmaceutically acceptable salts thereof are poor substrates for the above mentioned drug transporters.

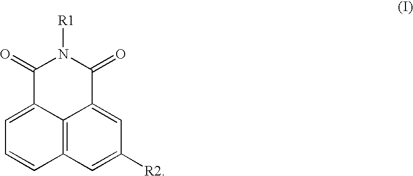

In formula (I):

R1 is —(CH₂)ₙNR3R4;

R2 is —OR5, halogen, —NR6R7, —NR6R7, sulphonic acid, nitro, —NR5COOR5, —NR5COR5 or —OCOR5;

R3 and R4 are independently H, C1-C4 alkyl group or, taken together with the nitrogen atom to which they are bonded, a non-aromatic nitrogen-containing heterocyclic group;

each R5 is independently H or a C1-C4 alkyl group;

R6 and R7 are independently H, a C1-C4 alkyl group or, taken together with the nitrogen atom to which they are bonded, a non-aromatic nitrogen-containing heterocyclic group; and

n is an integer from 0-3.

A compound of formula (I) may be protonated with a pharmaceutically acceptable salt at R1, or, when R2 is —NR6R7, R1, R2 or both. When protonated, a compound of formula (I) can form a salt with a pharmaceutically acceptable salt X. Preferably, the salt is a carboxylate anion of an organic carboxylic acid. Examples of suitable organic carboxylic acids are provided below.

Preferably in structural formula (I), n is 2; R3 and R4 are the same and are —H, —CH₃ or —CH₂CH₃; and R2 is —NO₂, —NH₂ or —NH₃⁺X⁻. More preferably, n is 2; R3 and R4 are —CH₃; and R2 is —NO₂, —NH₂ or —NH₃⁺X⁻. Suitable values for X⁻ are provided below.

More preferably, the compound of formula (I) is amonafide (Xanafide), represented by structural formula (II), or pharmaceutically acceptable salts thereof:

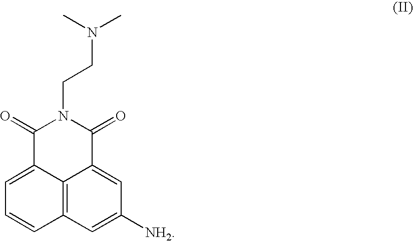

The compounds disclosed herein with two amine groups, including amonafide salts, can be monovalent, meaning that one of the amine groups is protonated, or divalent, meaning that both amine groups are protonated or a mixture thereof. A divalent compound can be protonated by two different monocarboxylic acid compounds (i.e., the two Xs in structural formula (I) represent two different monocarboxylic acid compounds), by two molar equivalents of the same monocarboxylic acid compound (i.e., the two Xs in structural formula (I) each represent one molar equivalent of the same monocarboxylic acid compound), or by one molar equivalent of a dicarboxylic acid compound (i.e., the two Xs in structural formula (I) together represent one dicarboxylic acid compound). Alternatively, three molar equivalents a divalent compound are protonated by two molar equivalents of a tricarboxylic acid compound. All of these possibilities are meant to be included within Structural Formulas (I) and (II) above.

The compounds of formula (I) can be administered as the free base or as a pharmaceutically acceptable salt. The term “pharmaceutically acceptable salt” means either an acid addition salt or a basic addition salt, whichever is possible to make with the compounds of the present invention. “Pharmaceutically acceptable acid addition salt” is any non-toxic organic or inorganic acid addition salt of the base compounds represented by formula (I) or formula (II). Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points. “Pharmaceutically acceptable basic addition salt” means non-toxic organic or inorganic basic addition salts of the compounds of formula (I) or formula (II). Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.

Preferably, a compound of formula (I) is administered as an organic carboxylic acid salt. An organic carboxylic acid is an organic compound having one or more carbon atoms and a carboxylic acid functional group. Suitable organic carboxylic acid compounds for use in preparing the compounds of the present invention are water soluble (typically a water solubility greater than 20% weight to volume), produce water soluble salts with aryl amines and alkyl amines and have a pKa>2.0. Included are aryl carboxylic acids, aliphatic carboxylic acids (typically C1-C4), aliphatic dicarboxylic acids (typically C2-C6), aliphatic tricarboxylic acids (typically C3-C8) and heteroalkyl carboxylic acids. An aliphatic carboxylic acid can be completely saturated (an alkyl carboxylic acid) or can have one or more units of unsaturation. A heteroalkyl carboxylic acid compound is an aliphatic carboxylic acid compound in which one or more methylene or methane groups are replaced by a heteroatom such as O, S, or NH. Examples of heteroalkyl carboxylic acid compounds include a C1-C5 heteroalkyl monocarboxylic acid compound (i.e., a C2-C6 alkyl monocarboxylic acid compound in which one methylene or methane group has been replaced with O, S or NH) and C3-C8 a heteroalkyl dicarboxylic acid compound (i.e., a C2-C7 alkyl dicarboxylic acid compound in which one methylene or methane group has been replaced with O, S or NH).

Examples of suitable organic acids are: saturated aliphatic monocarboxylic acids such as formic acid, acetic acid or propionic acid; unsaturated aliphatic monocarboxylic acids such as 2-pentenoic acid, 3-pentenoic acid, 3-methyl-2-butenoic acid or 4-methyl-3-pentenoic acid; functionalized acids such as hydroxycarboxylic acids (e.g. lactic acid, glycolic, pyruvic acid, mandelic acid); ketocarboxylic acids (e.g. oxaloacetic acid and alpha-ketoglutaric acid); amino carboxylic acids (e.g. aspartic acid and glutamic acid); saturated aliphatic dicarboxylic acids such as malonic acid, succinic acid or adipic acid; unsaturated aliphatic dicarboxylic acids such as maleic acid or fumaric acid; functionalized di- and tricarboxylic acids such as malic acid, tartaric acid, citric acid gluconic acid; aryl carboxylic acids having sufficient water solubility, e.g., 4-hydroxybenzoic acid, salicylic acid, anthranilic acid, anisic acid and vanillic acid.

Preferably, a compound of formula (I), including the compound of formula (II) forms a salt with malic acid or hydrochloric acid. Either mono- or divalent salts can be formed.

The term “aliphatic”, as used herein, means non-aromatic group that consists solely of carbon and hydrogen and may optionally contain one or more units of unsaturation, e.g., double and/or triple bonds. An aliphatic group may be straight chained or branched.

The term “alkyl”, as used herein, unless otherwise indicated, includes straight or branched saturated monovalent hydrocarbon radicals, typically C1-C10, preferably C1-C6. Examples of alkyl groups include, but are not limited to, methyl, ethyl, propyl, isopropyl, and t-butyl. Suitable substituents for a substituted alkyl include —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, where p and q are independently an integer from 1 to 6.

As used herein, the term “heteroalkyl” refers to an alkyl as defined above, in which one or more internal carbon atoms have been substituted with a heteroatom. Each heteroatom is independently selected from nitrogen, which can be oxidized (e.g., N(O)), secondary, tertiary or quaternized; oxygen; and sulfur, including sulfoxide and sulfone.

The term “aryl”, as used herein, refers to a carbocyclic aromatic group. Examples of aryl groups include, but are not limited to phenyl and naphthyl.

An aliphatic carboxylic acid compound can be straight or branched. An aliphatic carboxylic acid can be substituted (functionalized) with, one or more functional groups. Examples include a hydroxyl group (e.g., a hydroxy C2-C6 aliphatic monocarboxylic acids, a hydroxy C3-C8 aliphatic dicarboxylic acid and a hydroxy C4-C10 hydroxy aliphatic tricarboxylic acid), an amine (e.g., an amino C2-C6 aliphatic monocarboxylic acid, an amino C3-C8 aliphatic dicarboxylic acid and an amino C4-C10 aliphatic tricarboxylic acid), a ketone (e.g., a keto C2-C6 aliphatic monocarboxylic acid, a keto C3-C8 dicarboxylic acid or a keto C4-C10 tricarboxylic acid) or other suitable functional group.

Non-aromatic nitrogen-containing heterocyclic rings are non-aromatic nitrogen-containing rings which include zero, one or more additional heteroatoms such as nitrogen, oxygen or sulfur in the ring. The ring can be five, six, seven or eight-membered. Examples include morpholinyl, thiomorpholinyl, pyrrolidinyl, piperazinyl, piperidinyl, azetidinyl, azacycloheptyl, or N-phenylpiperazinyl.

The compounds disclosed herein are useful for the treatment of a subject. A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The compounds of the present invention can be used to treat a broad spectrum of cancers, including carcinomas, sarcomas and leukemias. Preferably, the compounds of formula (I) are employed to treat multi-drug resistant (MDR) cancers.

As used herein, multidrug resistance (MDR) refers to a state of cancer in which, having developed resistance to a single drug, cells also show cross-resistance to other structurally and mechanistically unrelated drugs. For example, a cancer that has developed MDR can show resistance to one or more of vinca alkaloids (vinblastine, vincristine and vinorelvine), one or more of the anthracyclines (doxorubicin, daunorubicin, epirubicin VP-16, idarubicin, and mitaxanthrone), to the RNA transcription inhibitor actinomycin-D or to the microtubule-stabilizing drug paclitaxel.

Methods of analyzing expression of the transporters that confer MDR and methods for detecting P-glycoprotein-associated multidrug resistance in patients' tumors are well known in the art. See, e.g. Beck et al., “Methods to detect P-glycoprotein-associated multidrug resistance in patients' tumors: consensus recommendations.”, Cancer Res. 2002 Jan. 15; 62(2):617; and Leith et al., “Correlation of Multidrug Resistance (MDR1) Protein Expression With Functional Dye/Drug Efflux in Acute Myeloid Leukemia by Multiparameter Flow Cytometry: Identification of Discordant MDR⁻/Efflux⁺ and MDR1⁺/Efflux⁻ Cases”, Blood, Vol. 86, No 6 (September 15), 1995: pp 2329-2342. The teachings of both publications are incorporated hereby by reference. Example 1 below provides further details on methods of detecting MDR-cancers.

Examples of multidrug-resistant carcinomas, including adenocarcinomas that can be treated using the compounds of the present invention are esophageal, breast, colon, lung, kidney and prostate cancers. An example of multidrug-resistant resistant sarcomas that can be treated using the compounds of the present invention are gliomas. Examples of multidrug-resistant leukemias that can be treated using the method include Acute Myelogenous Leukemia (AML), Chronic Myelogenous Leukemia (CML), Acute Lymphocytic Leukemia (ALL) and Chronic Lymphocytic Leukemia (CLL) and chronic prolymphocytic leukemias.

Other examples of the multidrug-resistant cancers that are treated using the compounds of the present invention include colon, kidney, adrenocortical and hepatocellular cancers, breast tumors, ovarian cancer, Leukemia, Non-Small Cell Lung, small-cell lung cancer, Colon, CNS, Melanoma, Ovarian, Renal, Prostate and Breast cancers. More preferably, the multidrug-resistant cancer being treated is refractory leukemia. As used herein, the term “refractory leukemia” refers to leukemia (including all the subtypes identified above) in which the high level of white blood cells is not decreasing in response to treatment. In another embodiment, the compounds of formula (I) are used to treat a relapsed leukemia, which is a type of multidrug-resistant leukemia (including all subtypes identified above) which no longer responds to treatment to which it responded previously.

In one embodiment, the cancer is any of the cancer described in the two preceding paragraphs and is resistant to the treatment by daunorubicin. In another embodiment, the cancer is any of the cancer described in the two preceding paragraphs and is resistant to the treatment by idarubicin. In another embodiment, the cancer is any of the cancer described in the two preceding paragraphs and is resistant to the treatment by Ara-C. In another embodiment, the cancer is any of the cancer described in the two preceding paragraphs and is resistant to the treatment by etoposide. In another embodiment, the cancer is any of the cancer described in the two preceding paragraphs and is resistant to the treatment by mitoxantrone. In another embodiment, the cancer is any of the cancer described in the two preceding paragraphs and is resistant to the treatment by liposomal daunorubicin. In another embodiment, the cancer is any of the cancer described in the two preceding paragraphs and is resistant to the treatment by 6-thioguanine. In another embodiment, the cancer is any of the cancer described in the two preceding paragraphs and is resistant to the treatment by cladrabine. In another embodiment, the cancer is any of the cancer described in the two preceding paragraphs and is resistant to the treatment by clofarabine. In another embodiment, the cancer is any of the cancer described in the two preceding paragraphs and is resistant to the treatment by vincristine. In another embodiment, the cancer is any of the cancer described in the two preceding paragraphs and is resistant to the treatment by adriamycin. In another embodiment, the cancer is any of the cancer described in the two preceding paragraphs and is resistant to the treatment by doxorubicin (B). In another embodiment, the cancer is any of the cancer described in the two preceding paragraphs and is resistant to the treatment by vinblastine. In another embodiment, the cancer is any of the cancer described in the two preceding paragraphs and is resistant to the treatment by vinorelvine. In another embodiment, the cancer is any of the cancer described in the two preceding paragraphs and is resistant to the treatment by epirubicin VP-16. In another embodiment, the cancer is any of the cancer described in the two preceding paragraphs and is resistant to the treatment by actinomycin-D. In another embodiment, the cancer is any of the cancer described in the two preceding paragraphs and is resistant to the treatment by paclitaxel (or another taxane such as docetaxel). In another embodiment, the cancer is any of the cancer described in the two preceding paragraphs and is resistant to the treatment by colchicine. In another embodiment, the cancer is any of the cancer described in the two preceding paragraphs and is resistant to the treatment by digoxin. In another embodiment, the cancer is any of the cancer described in the two preceding paragraphs and is resistant to the treatment by saquinivir. In another embodiment, the cancer is any of the cancer described in the two preceding paragraphs and is resistant to the treatment by rhodamine. In another embodiment, the cancer is any of the cancer described in the two preceding paragraphs and is resistant to the treatment by sulfinpyrazone. In another embodiment, the cancer is any of the cancer described in the two preceding paragraphs and is resistant to the treatment by nucleoside monophosphates. In another embodiment, the cancer is any of the cancer described in the two preceding paragraphs and is resistant to the treatment by topotecan. In another embodiment, the cancer is any of the cancer described in the two preceding paragraphs and is resistant to the treatment by CPT-11. In another embodiment, the cancer is any of the cancer described in the two preceding paragraphs and is resistant to the treatment by prednisone. In another embodiment, the cancer is any of the cancer described in the two preceding paragraphs and is resistant to the treatment by L-asparginase. In another embodiment, the cancer is any of the cancer described in the two preceding paragraphs and is resistant to the treatment by methotrexate. In another embodiment, the cancer is any of the cancer described in the two preceding paragraphs and is resistant to the treatment by 6-Mercaptopurine (6-MP). In another embodiment, the cancer is any of the cancer described in the two preceding paragraphs and is resistant to the treatment by cyclophosphamide. In another embodiment, the cancer is any of the cancer described in the two preceding paragraphs and is resistant to the treatment by chlorambucil. In another embodiment, the cancer is any of the cancer described in the two preceding paragraphs and is resistant to the treatment by hyroxyurea. In another embodiment, the cancer is any of the cancer described in the two preceding paragraphs and is resistant to the treatment by busulfan. In another embodiment, the cancer is any of the cancer described in the two preceding paragraphs and is resistant to the treatment by any combination of two or more pharmaceutically active ingredients described in the instant paragraph.

In one embodiment, the cancer is any of the cancer described in the three preceding paragraphs and the resistance is mediated by an ABC transporter. In one embodiment, the cancer is any of the cancer described in the three preceding paragraphs and the resistance is mediated by ABCB1 transporter. In one embodiment, the cancer is any of the cancer described in the three preceding paragraphs and the resistance is mediated by ABCC1 transporter. In one embodiment, the cancer is any of the cancer described in the three preceding paragraphs and the resistance is mediated by ABCC2 transporter. In one embodiment, the cancer is any of the cancer described in the three preceding paragraphs and the resistance is mediated by ABCC3 transporter. In one embodiment, the cancer is any of the cancer described in the three preceding paragraphs and the resistance is mediated by ABCC4 transporter. In one embodiment, the cancer is any of the cancer described in the three preceding paragraphs and the resistance is mediated by ABCC5 transporter. In one embodiment, the cancer is any of the cancer described in the three preceding paragraphs and the resistance is mediated by ABCG2 transporter.

An “effective amount” is the quantity of compound in which a beneficial clinical outcome is achieved when the compound is administered to a subject with a multi-drug resistant cancer. A “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of cancer. The skilled artisan will be able to determine appropriate dosages depending on these and other factors. Effective amounts of the disclosed compounds for therapeutic application typically range between about 0.35 millimoles per square meter of body surface area (mmole/msq) per day and about 2.25 mmole/msq per day, and preferably between 1 mmole/msq and 1.5 mmole/msq on five day cycles by intravenous infusion.

The disclosed compounds can be administered alone or in combination with other pharmaceutical agents. Examples of pharmaceutical agents that can be used in combination with the compounds of formula (I) are: colchicine, doxorubicin, VP16 (etoposide), adriamycin, vinblastine, digoxin, saquinivir, paclitaxel; verapamil, PSC833, GG918, V-104, Pluronic L61; daunorubicin, vincristine, rhodamine; cyclosporin A, V-104; sulfinpyrazone; methotrexate; nucleoside monophosphates; mitoxantrone, topotecan, CPT-11, fumitremorgin C, and GF120918.

The disclosed compounds are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), or rectally, depending on the type of cancer to be treated. Oral or parenteral administration are preferred modes of administration.

The disclosed compounds can be administered to the subject in conjunction with an acceptable pharmaceutical carrier as part of a pharmaceutical composition for treatment of cancer. Formulation of the compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrasn) are known in the art (Baker, et al., “Controlled Release of Biological Active Agents”, John Wiley and Sons, 1986).

### EXEMPLIFICATION

### Example 1

### Method of Detecting P-glycoprotein-associated Multidrug Resistance in Patients' Tumors

### Analysis of MDR1 Expression and Functional Efflux.

Blasts from pretreatment bone marrow/peripheral blood samples are enriched by density gradient separation and assays are performed either on fresh cells or after cryopreservation and thawing. MDR1 expression by leukemic blasts is measured using the MDR1-specific antibody MRK16 (Kamiya, Thousand Oaks, Calif.) in three-color flow cytometric assays where blasts are co-stained with MRK16, the hematopoietic stem/progenitor cell antigen CD34, and the pan-myeloid antigen CD33, as previously described in Leith et al., Blood, Vol. 86, No 6 (September 15), 1995: pp 2329-2342. Appropriately matched isotype controls are used in all assays. To assess functional drug efflux and correlate efflux with MDR1 expression, the ability of leukemic blasts to efflux a fluorescent dye, DiOC2, is measured in single-color flow cytometric assays. The fluorescent dye, DiOC2, is an MDR1 substrate, but unlike other MDR1 substrates such as doxorubicin and Rhodamine 123, it does not appear to be transported by the multidrug resistance protein (MRP), one of the more recently identified drug transporters, and thus may be more specific than these other drugs/dyes for MDR1-mediated transport.

Briefly, leukemic blasts are incubated in media containing DiOC2 to allow uptake for 30 minutes; the blasts are then washed, baseline dye uptake measured, and resuspended in fresh dye-free media with or without the MDR1-modulator cyclosporine A (CsA; 2500 ng/mL; Sandoz Pharmaceuticals, Basel, Switzerland) and incubated for 90 minutes at 37° C. to allow efflux. Cells are then resuspended in fresh 4° C. media for immediate flow cytometric analysis. The MDR1(+) DOX cell lines and MDR1(—parental line are used as controls in all experiments.

### Analysis of MDR1 Expression and Efflux Data.

Analyses are performed on a FACScan flow cytometer using Lysis II software (Becton Dickinson, Thousand Oaks, Calif.). MRK16 staining of gated leukemic blasts compared with control cells is measured using the Kolmogorov-Smirnov (KS) statistic, denoted D, which measures the difference between two distribution functions and generates a value ranging from −1.0 to 1.0. This method accurately identifies small differences in fluorescence and is useful in detection of low level MDR1 expression, which frequently occurs in primary patient samples. MRK16 staining intensity is categorized for descriptive purposes as follows: bright (D 0.25), moderate (0.15 D>0.25), dim (0.10 D<0.15), and negative (D<0.10); however, correlations with clinical outcome are largely performed using the D value as a continuous variable. DiOC2 efflux is assessed by analyzing cellular fluorescence of gated leukemic blasts after efflux in the presence/absence of CsA; differences in fluorescence were analyzed with KS statistics and a D value of 0.25 is used to define a case as efflux (+).

### Example 2

### Amonafide Activity in K562 and K562 Resistant Cell Lines

Amonafide was tested in cell proliferation (MTS) assays in K562 (human leukemia) cell lines and a K562 cell line selected for resistance to daunorubicin. The K562 resistant cell line has been characterized with over-expression of the multidrug resistant protein (ABCB1, PGP). Known substrates for ABCB1 (daunorubicin, doxorubicin, idarubicin, mitoxantrone and etoposide) were used as controls.

### MTS Cell Proliferation Assay

MTS assay is an assay in which the bioreduction of the MTS reagent (3-(4,5-dimethylthiazol-2-yl)-5-(3-carboxymethoxyphenyl)-2-(4-sulfophenyl)-2H-tetrazolium)) by cells is being measured to assess metabolic activity of cells is based on the ability of a mitochondrial dehydrogenase enzyme from viable cells to cleave the tetrazolium rings of the MTS and form formazan crystals which are largely impermeable to cell membranes, thus resulting in its accumulation within healthy cells. Solubilization of the cells by the addition of a detergent results in the liberation of the crystals which are solubilized. The number of surviving cells is directly proportional to the level of the formazan product created. The color can then be quantified using a simple colorimetric assay. The results can be read on a multiwell scanning spectrophotometer (ELISA reader).

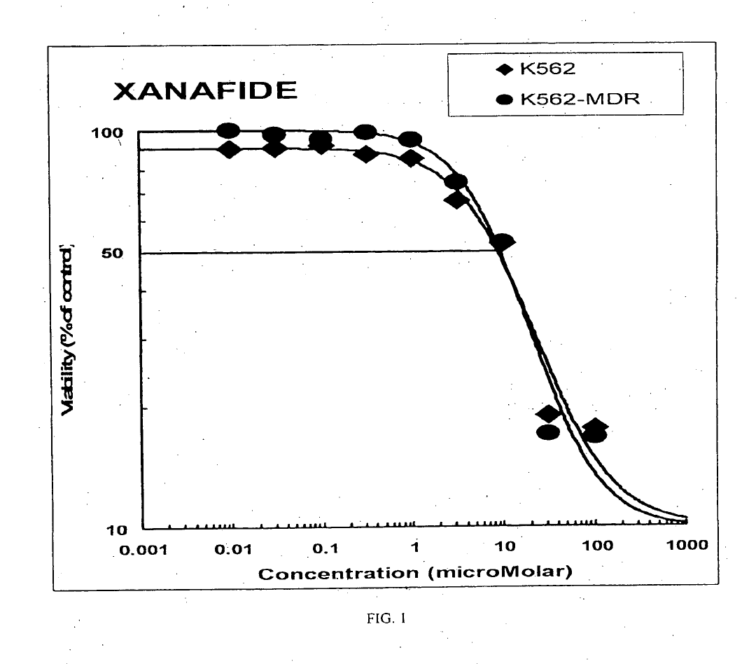

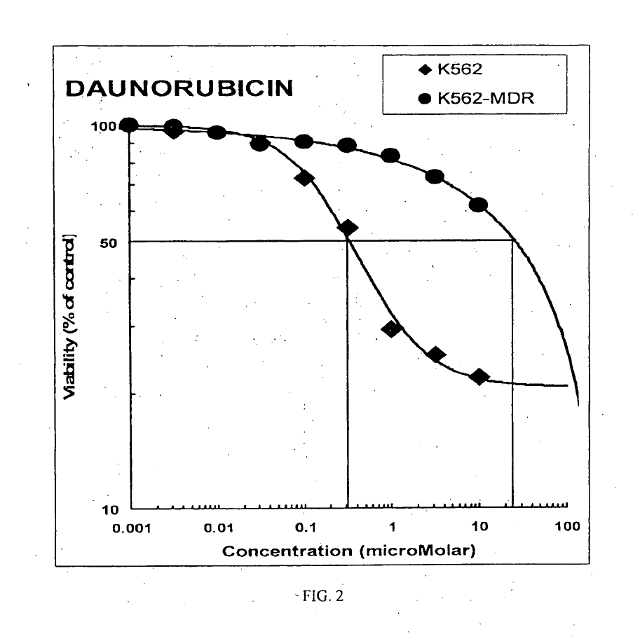

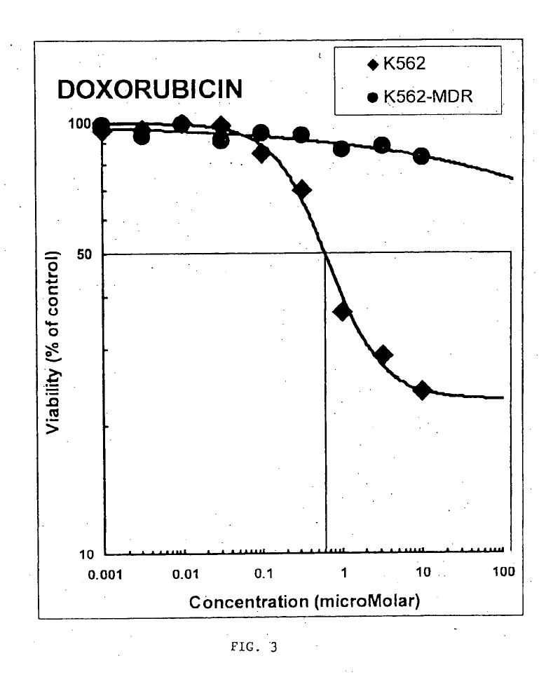

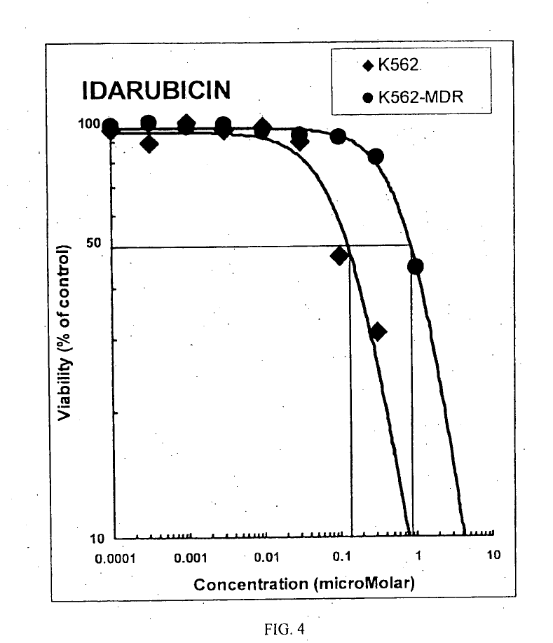

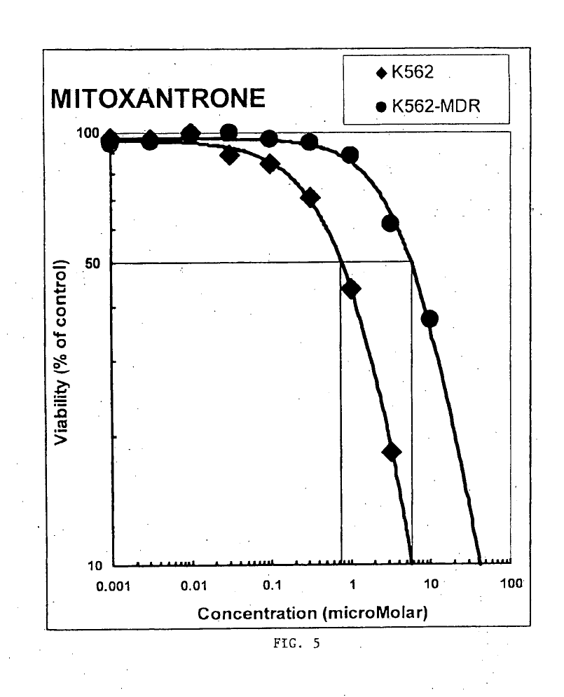

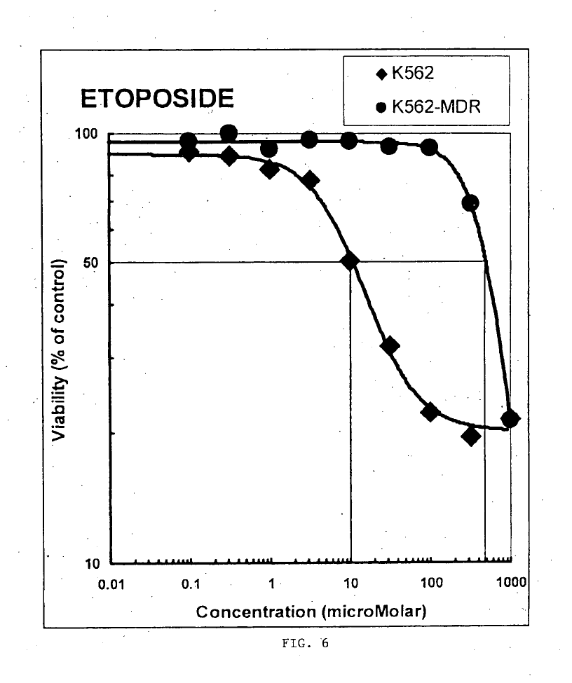

The results are presented in FIG. 1, FIG. 2, FIG. 3, FIG. 4, FIG. 5 and FIG. 6 as percent of the untreated control. The LC₅₀ for the control drugs (daunorubicin, doxorubicin, idarubicin, etoposide, and mitoxantrone) was increased by 1 to 2 log units. In contrast, amonafide was equipotent in both cell lines.

### Example 3

### Amonafide Activity in P388 and P388 Resistant Cell Lines

Amonafide was tested in cell proliferation (MTT) assays in P388 (murine leukemia) cell lines and a P388 cell line selected for resistance to doxorubicin. The P388 resistant cell line has been characterized with over-expression of the multidrug resistant protein (MDR; p-glycoprotein). The over-expression ratio (level of MDR in resistant cell line over level of MDR in the parental cell line) as determined by PCR is 19 fold and as determined by microarray, 148 fold. Daunorubicin, which is a known substrate for pgp, was used as a control.

### MTT Cell Proliferation Assay

MTT [3-(4,5-dimethylthiazol-2-yl)-2,5-diphenyltetrazolium bromide] assay, first described by Mosmann in 1983, is based on the ability of a mitochondrial dehydrogenase enzyme from viable cells to cleave the tetrazolium rings of the pale yellow MTT and form a dark blue formazan crystals which is largely impermeable to cell membranes, thus resulting in its accumulation within healthy cells. Solubilization of the cells by the addition of a detergent results in the liberation of the crystals which are solubilized. The number of surviving cells is directly proportional to the level of the formazan product created. The color can then be quantified using a simple colorimetric assay. The results can be read on a multiwell scanning spectrophotometer (ELISA reader).

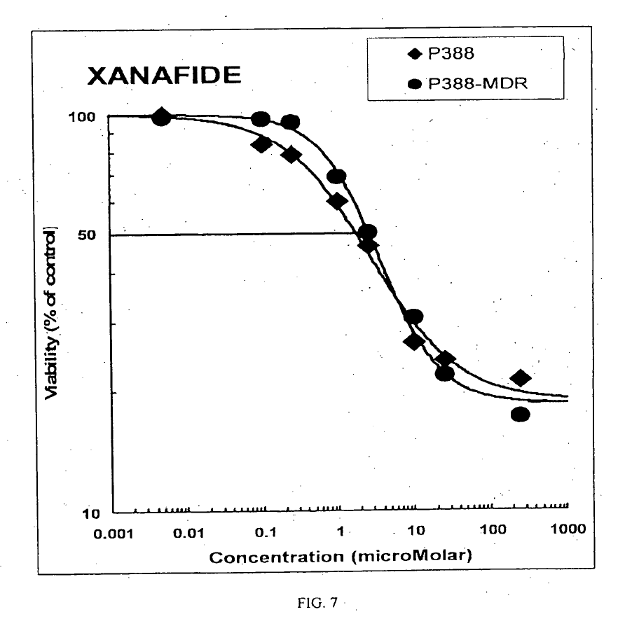

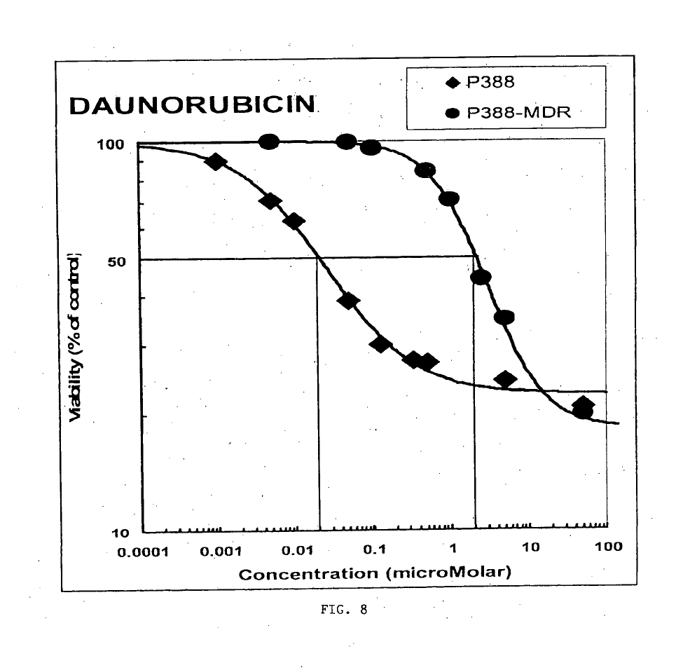

The results are presented in FIG. 7 and FIG. 8 as percent of the untreated control. The LC₅₀ for the control drug (daunorubicin) was increased by 2 log units. In contrast, amonafide was equipotent in both cell lines

### Example 4

### Amonafide Activity in MCF7 and MCF7 Resistant Cell Lines

Amonafide was tested in cell proliferation (clonogenic) assays in MCF7 (human breast cancer) cell lines and a MCF7 cell line selected for resistance to doxorubicin. The MCF7 resistant cell line has been characterized with over-expression of the multidrug resistant protein (MDR; p-glycoprotein). The over-expression ratio (level of MDR in resistant cell line over level of MDR in the parental cell line) as determined by PCR is 29 fold and as determined by microarray, 231 fold. Daunorubicin and doxorubicin, which are both known substrate for MDR, were used as a control.

### Clonogenic Assay

In the clonogenic assay cells are treated in vitro and then suspended in a soft agar/cell media mixture. The cells are allowed to grow in definable and distinct clumps of cells, referred to as clones, based upon the survival of the cells originally treated. After a defined time span the number of clones is then counted by microscope. The lower the number of clumps of cells the higher the efficacy of the original treatment for killing the cells.

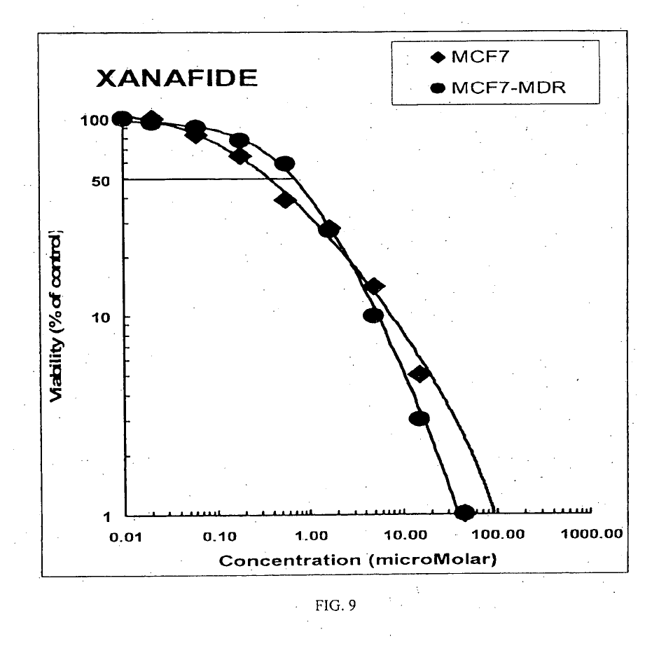

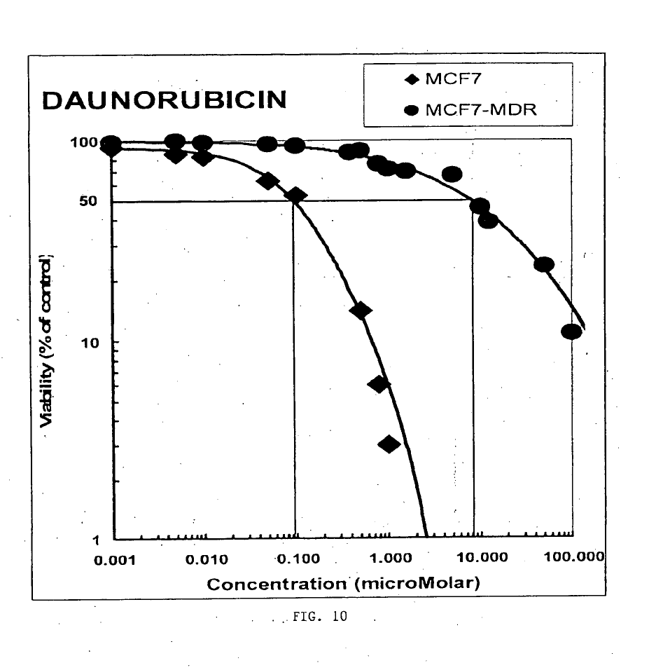

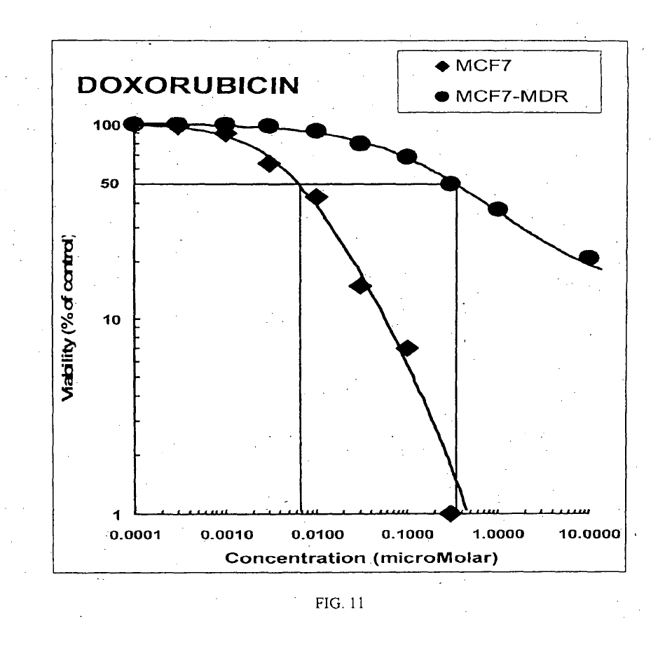

The results are presented in FIG. 9, FIG. 10 and FIG. 11 as percent of the untreated control. The LC₅₀ for the control drug (daunorubicin) was increased by 2 log units. In contrast, amonafide was equipotent in both cell lines

### Example 5

### Amonafide Activity in IGROV1 and IGROV1 Resistant Cell Lines

Amonafide was tested in cell proliferation (SRB) assays in IGROV 1 (human ovarian) cell lines and IGROV1-T8, a cell line selected for resistance to topotecan. The T8 resistant cell line has been characterized with over-expression of the multidrug resistant protein BCRP (breast cancer resistance protein). Topotecan, which is a known substrate for BCRP, was used as a control.

### SRB Cell Proliferation Assay

Sulforhodamine-B (SRB) assay is performed to assess cell survival. SRB is a water-soluble dye that binds to the basic amino acids of the cellular proteins. Thus, colorimetric measurement of the bound dye provides an estimate of the total protein mass that is related to the cell number. The color can then be quantified using a simple colorimetric assay. The results can be read on a multiwell scanning spectrophotometer (ELISA reader).

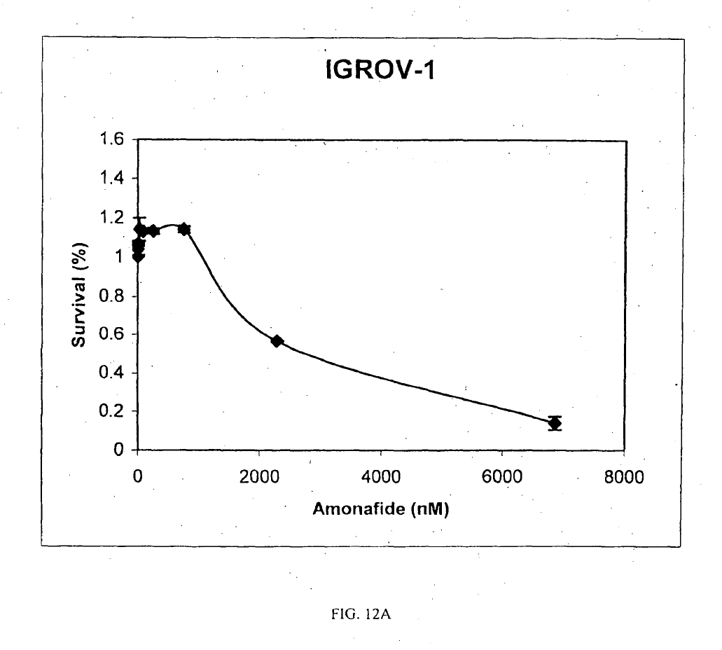

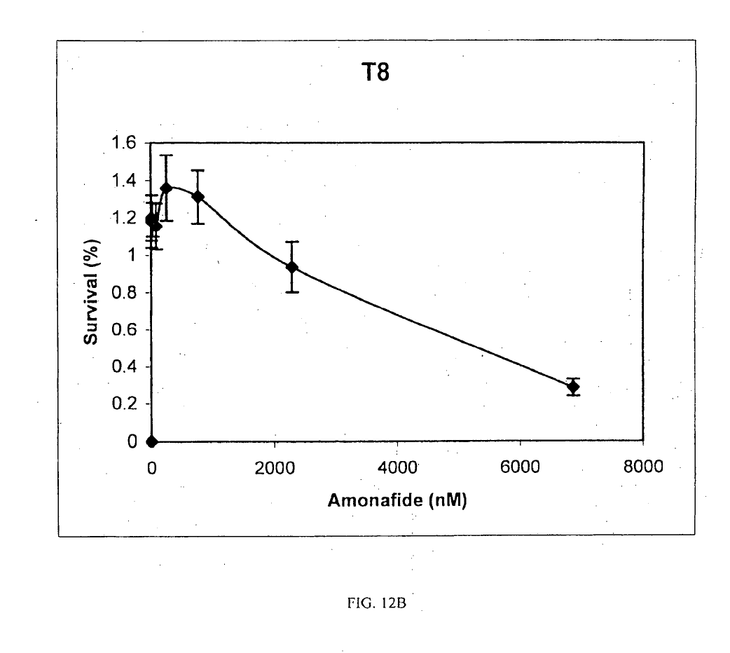

The results are presented in Table 2 and FIG. 12A (for IGROV-1) and FIG. 12B (for IGROV1-T8) and as percent of the untreated control. The T8 resistant line was 10 fold more resistant to Topotecan than to Amonafide

TABLE 2
Activity of Amonafide and Topotecan in an ovarian cancer cell line
(IGROV1) and a resistant subline (T8) with increased BCRP expression

| | IC₅₀ (nM) | | | | | |
|---|---|---|---|---|---|---|
| | Amonafide | Amonafide | Amonafide | Amonafide | TPT | TPT |
| Cell line | (1) | (2) | (3) | (average) | (1) | (2) |
| IGROV1 | 2544 | 3729 | 2529 | 2934 | 36.0 | 73.9 |
| T8 | 5182 | 4871 | 5833 | 5295 | 1100 | 1091 |

### Example 6

### Amonafide Activity in HL60 and HL60NCR Resistant Cell Lines

Amonafide was tested in cell proliferation (WST-1) assays in HL60 (human promyelocytic leukemia) cell lines and HL60/VCR, a cell line selected for resistance to vincristine. The HL60NCR resistant cell line has been characterized with over-expression of the multidrug resistant protein (MDR; p-glycoprotein). The over-expression ratio (level of MDR in resistant cell line over level of MDR in the parental cell line) is 8 fold with a 10 fold increase in pgp function. Pgp surface expression was measured by flow cytometry with the MRK16 antibody and Pgp function with the generic substrate, Rh123. Daunorubicin, doxorubicin, and mitoxantrone which are known substrates for MDR, were used as a control.

### WST-1 Cell Proliferation Assay

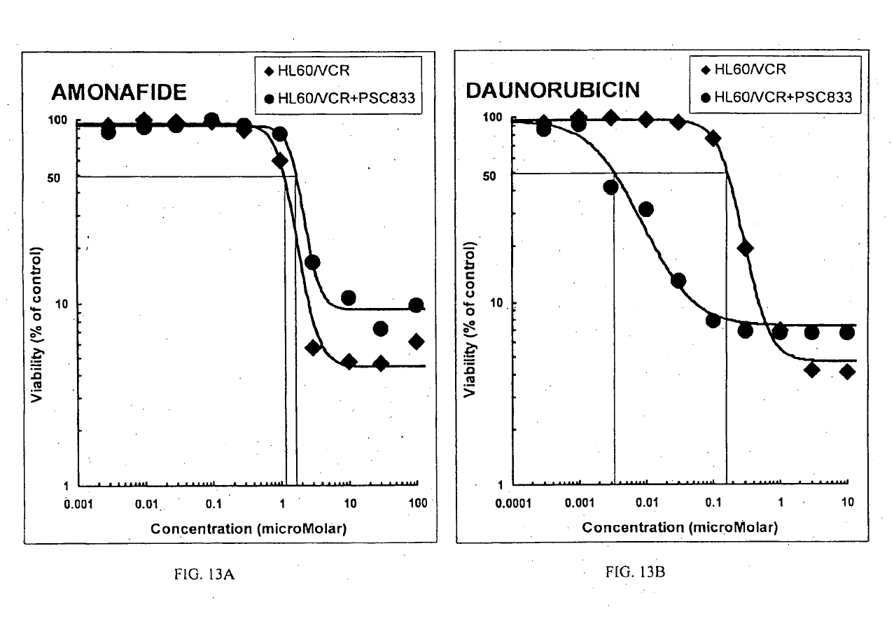

WST-1 is a water-soluble tetrazolium salt that can be used for cell proliferation or cell viability assays. The rate of WST-1 cleavage by mitochondrial dehydrogenases correlates with the number of viable cells in the culture. WST-1 is added directly to the cells ( 1/10th of the culture volume) and absorbance at 450 nm can be measured using an ELISA plate reader following a short incubation at 37° C.
The results are presented in Table 3 and FIG. 13A and FIG. 13B. HL60/VCR cells were resistant to the Topo II drugs. Amonafide is equipotent in both cell lines and amonafide cytotoxicity is unaffected by the Pgp inhibitor, PSC833. Daunorubicin is 2 log units less potent in the Pgp+ line.

TABLE 3
Comparison of IC50 values of amonafide, daunorubicin,
doxorubicin, and mitoxantrone in wild-type and MDR-expressing
HL60 acute promyelocytic leukemia cells.

| | HL60 | HL60/MDR | Fold Change |
|---|---|---|---|
| | μM | μM | in IC₅₀ |
| Amonafide | 6.98 | 2.04 | 0.29 |
| Daunorubicin | 0.015 | 0.092 | 6.13 |
| Doxorubicin | 0.033 | 0.161 | 4.87 |
| Mitoxantrone | 0.008 | 3.114 | 390.7 |

### Example 7

### Amonafide Activity in HL60 and HL60/ADR Resistant Cell Lines

Amonafide was tested in cell proliferation (WST-1) assays in HL60 (human promyelocytic leukemia) cell lines and HL60/ADR, a cell line selected for resistance to adriamycin. The HL60/ADR resistant cell line has been characterized with over-expression of the multidrug resistant protein (MRP-1). There is an 8-10 fold increase in functional expression of MRP1 (multidrug resistance protein) in this HL60/ADR cell line.

MRP-1 surface expression was measured by flow cytometry with the MRPm6 antibody. Daunorubicin a known substrates for MRP-1, was used as a control.

### WST-1 Cell Proliferation Assay

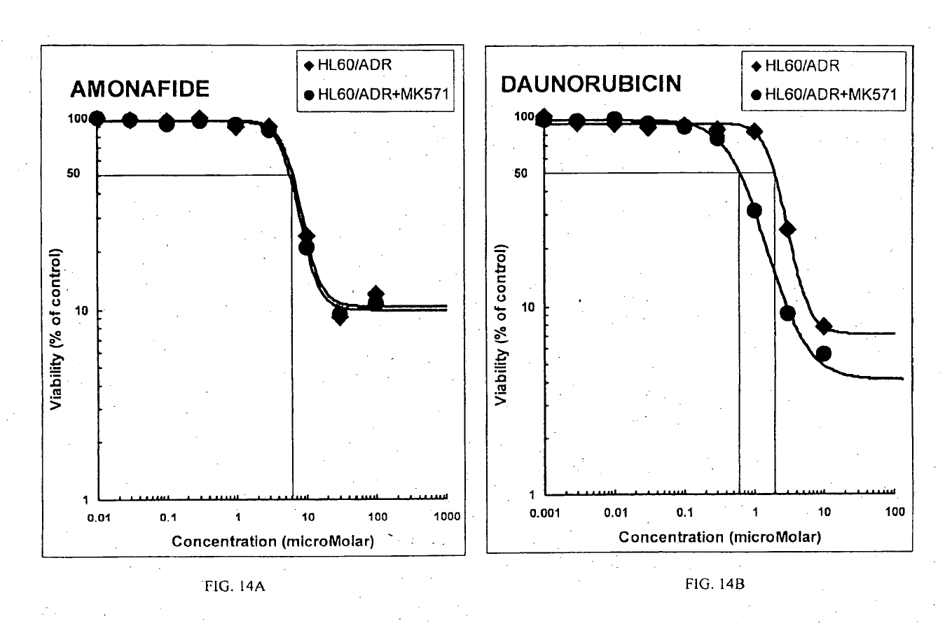

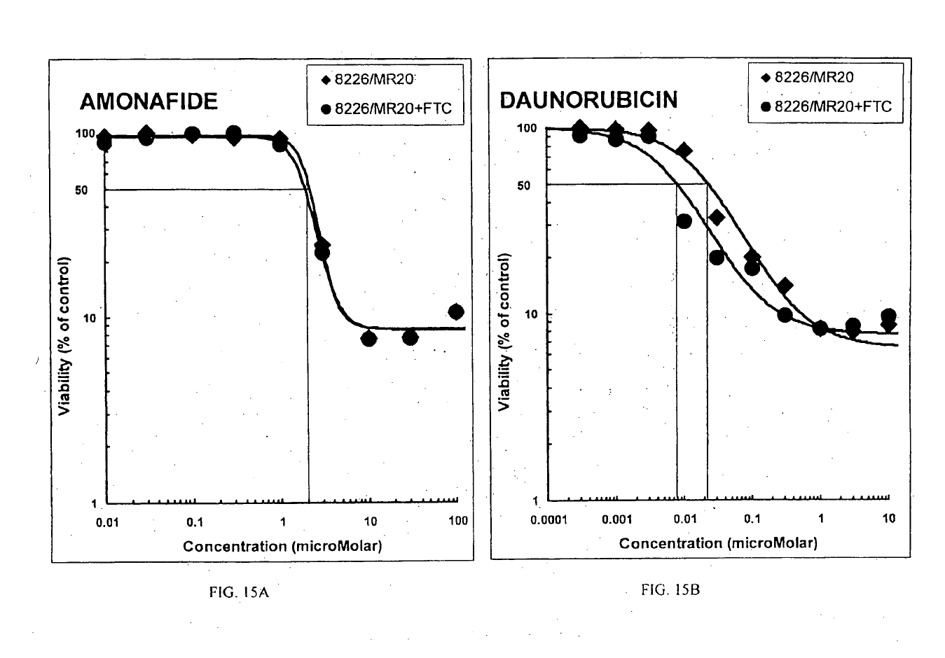

WST-1 is a water-soluble tetrazolium salt that can be used for cell proliferation or cell viability assays. The rate of WST-1 cleavage by mitochondrial dehydrogenases correlates with the number of viable cells in the culture. WST-1 is added directly to the cells ( 1/10th of the culture volume) and absorbance at 450 nm can be measured using an ELISA plate reader following a short incubation at 37° C.
The results are presented in FIG. 14A and FIG. 14B. HL60/ADR cells were resistant to the daunorubicin. Amonafide is equipotent in both cell lines and amonafide cytotoxicity is unaffected by the MRP1 inhibitor, MK571. Daunorubicin is ½ log unit less potent in the MRP1+ line.

### Example 8

### Amonafide Activity in 8226 and 8226/MR20 Resistant Cell Lines

Amonafide was tested in cell proliferation (WST-1) assays in 8226 (human myeloma) cell lines and 8226/MR20, a cell line selected for resistance to mitoxantrone. The 8226/MR20 resistant cell line has been characterized with over-expression of the multidrug resistant protein (BCRP). There is an 2 fold increase in functional expression of BCRP (breast cancer resistant protein) in this 8226/MR20 cell line.

BCRP surface expression was measured by flow cytometry with the BXP21 antibody. Daunorubicin a known substrates for BCRP, was used as a control.

### WST-1 Cell Proliferation Assay

WST-1 is a water-soluble tetrazolium salt that can be used for cell proliferation or cell viability assays. The rate of WST-1 cleavage by mitochondrial dehydrogenases correlates with the number of viable cells in the culture. WST-1 is added directly to the cells ( 1/10th of the culture volume) and absorbance at 450 nm can be measured using an ELISA plate reader following a short incubation at 37° C.

The results are presented in FIG. 15A and FIG. 15B. 8226/MR20 cells were resistant to the daunorubicin. Amonafide is equipotent in both cell lines and amonafide cytotoxicity is unaffected by the BCRP inhibitor, Fumitremorgin C. Daunorubicin is ½ log unit less potent in the BCRP line.

### Example 9

### Resistance Ratios

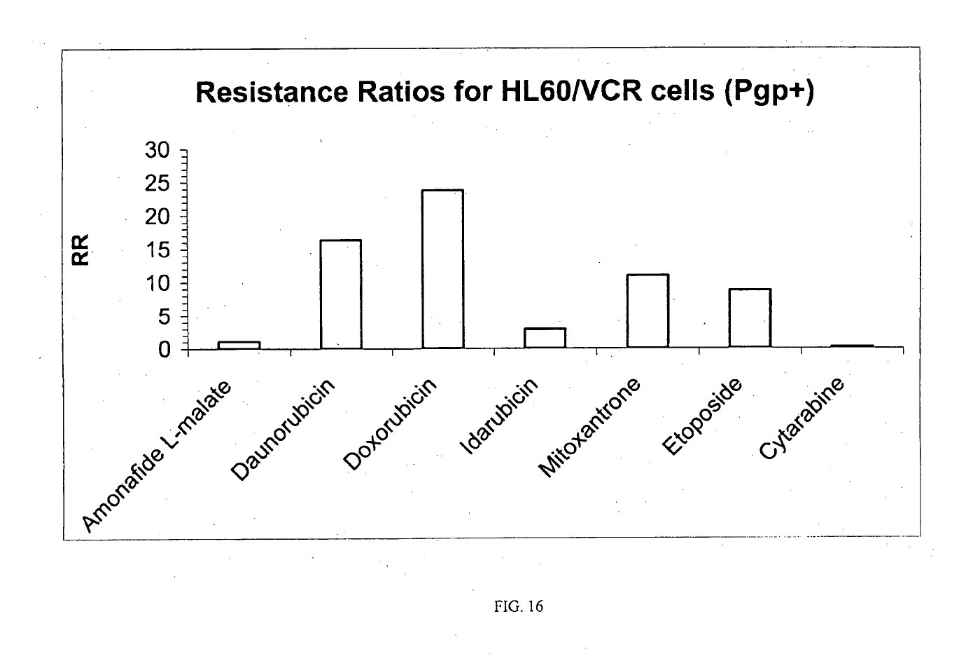

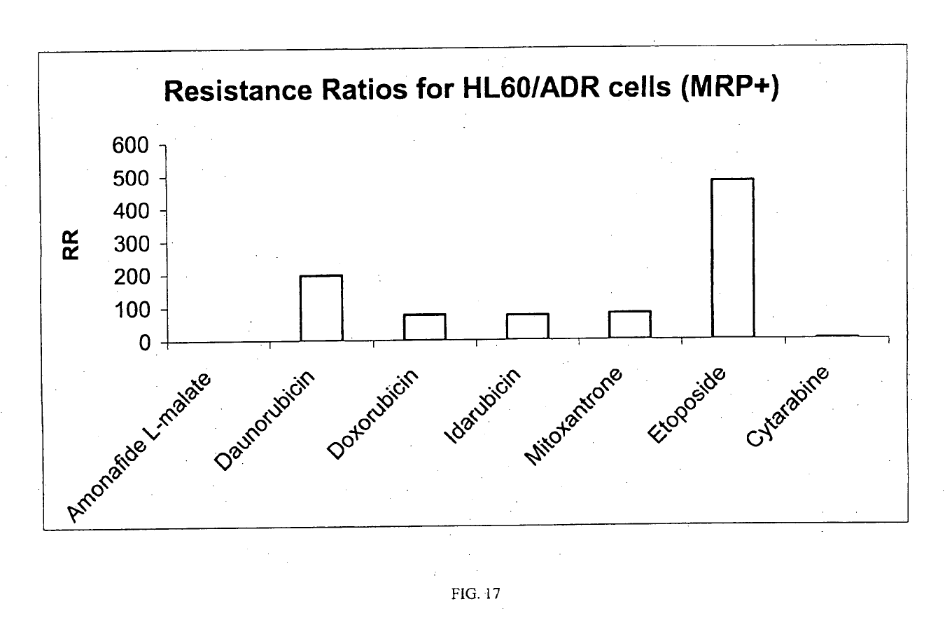

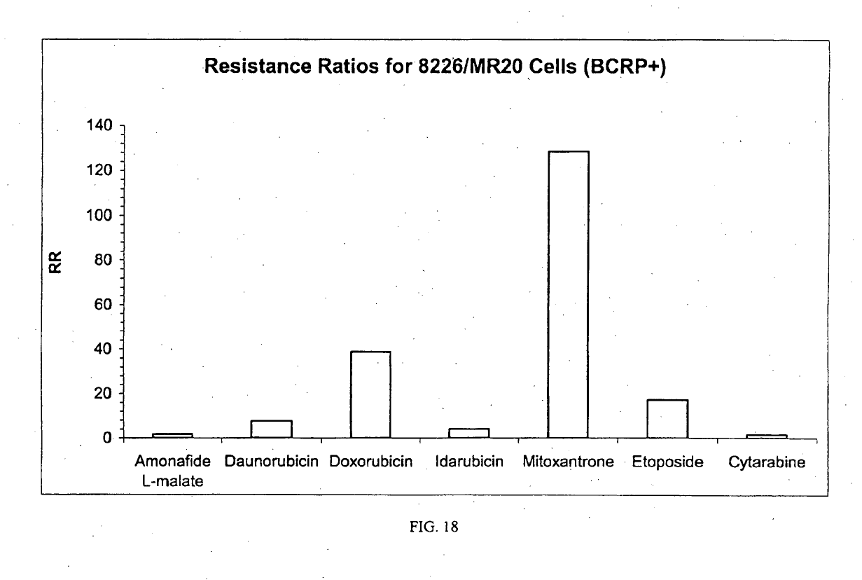

The survival data from Examples 6, 7 and 8 were used to calculate Resistance Ratios for amonafide and other cytotoxic drugs in the three pairs of parental and resistant cell lines (HL60-HL60VCR/HL60-HL60ADR/8226-8226/MR20). The Resistance Ratios were calculated as IC₅₀ of the resistant cell line/IC₅₀ of the parental cell line. The resistance ratios for the three paired lines are plotted in FIG. 16, FIG. 17 and FIG. 18.

The results demonstrate that amonafide is not a substrate for Pgp, MRP-1 or BCRP.

### Example 10

### Resistance Modifying Factors

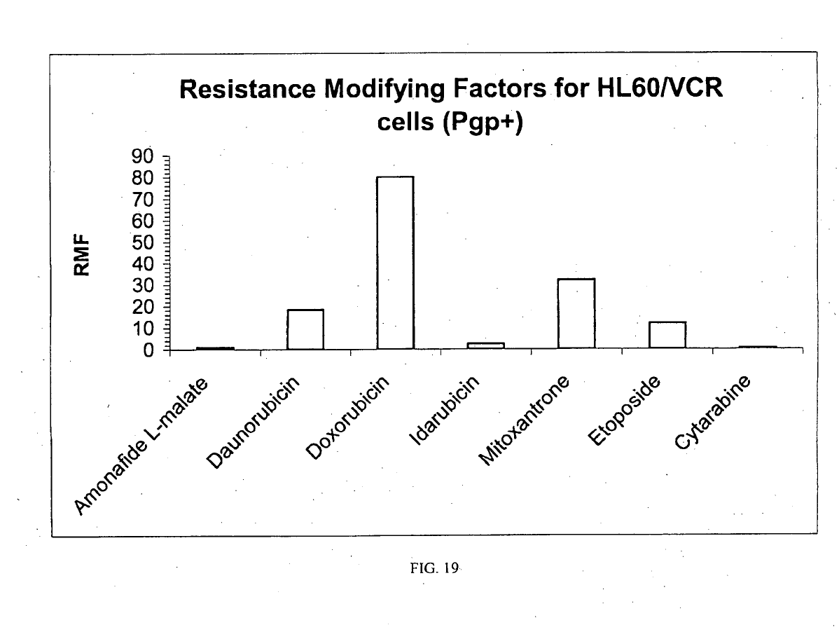

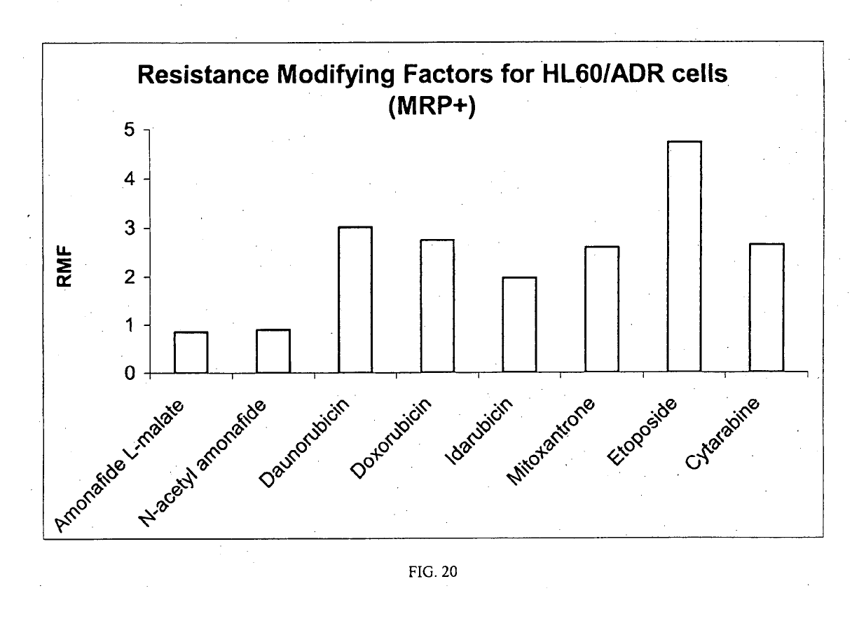

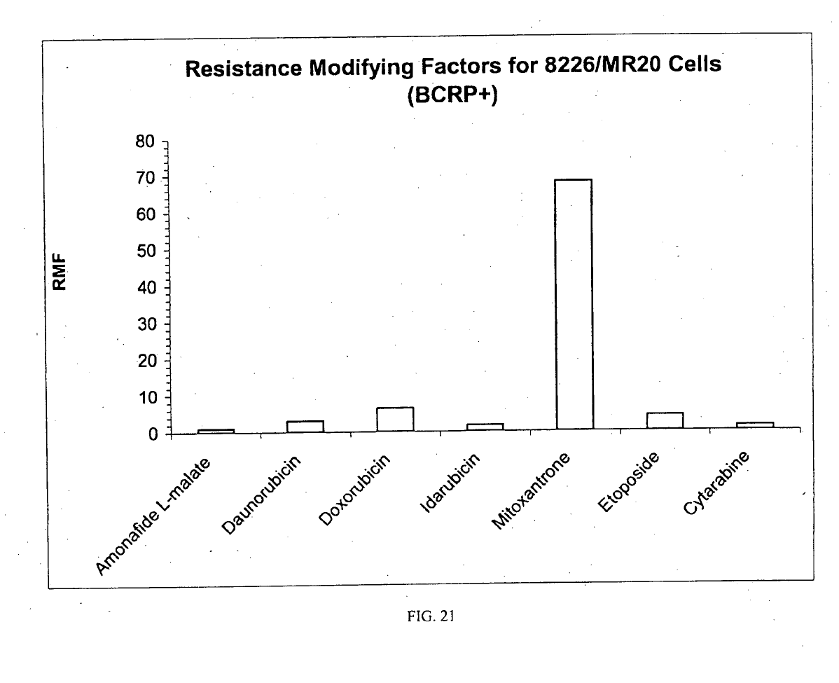

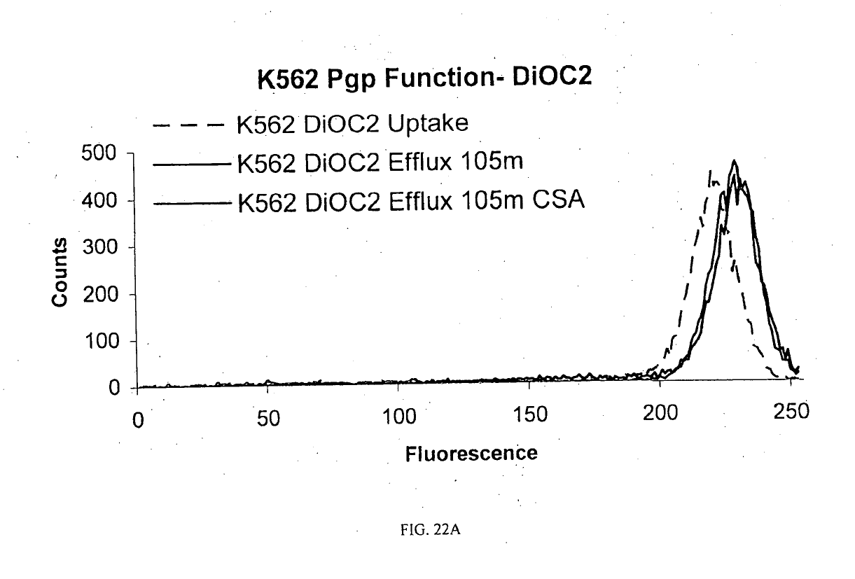

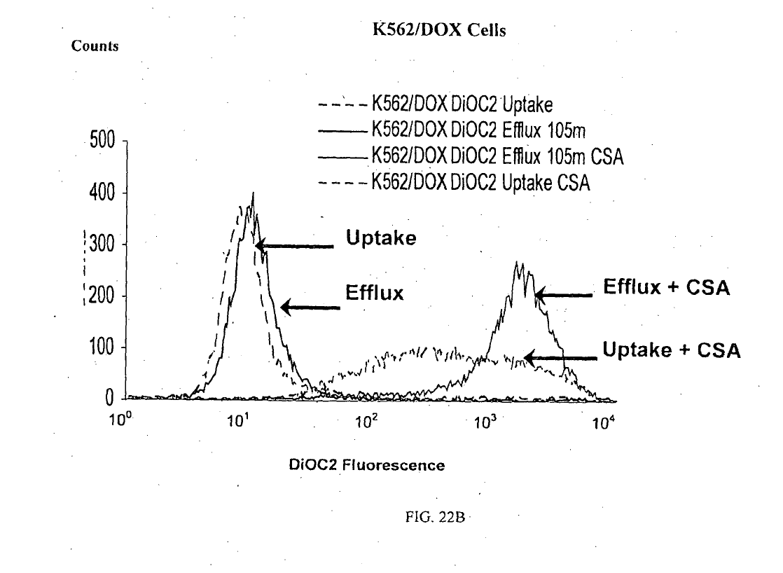

The survival data from Examples 6, 7 and 8 were used to calculate Resistance Modifying Factors for amonafide and other cytotoxic drugs in the three pairs of parental and resistant cell lines (HL60-HL60VCR/HL60-HL60ADR/8226-8226/MR20). The Resistance Modifying Factors were calculated as IC₅₀ of the resistant cell line in the absence of modulator/IC₅₀ of the resistant cell line in the presence of modulator. The Resistance Modifying Factors for the three paired lines are plotted in FIG. 19, FIG. 20 and FIG. 21.

The results demonstrate that amonafide is not a substrate for Pgp, MRP-1 or BCRP.

### Example 11

### Amonafide Retention in K562 and K562/DOX Cells

The uptake and efflux of amonafide and a control Pgp substrate (DiOC2) were measured in the paired Pgp negative and Pgp positive cell lines, K562-K562/DOX

### Functional Efflux Assay.

DiOC2, a fluorescent substrate of Pgp, was used to measure Pgp-mediated efflux. Cells were incubated in medium containing DiOC2 (presence or absence of CSA). The cells were washed with PBS and resuspended in medium. An aliquot was taken for cytometric quantitation of baseline dye uptake. The remaining samples were incubated again either with or without CSA and resuspended in fresh, chilled medium in order to assess dye efflux also by flow cytometric analysis.

### Drug Accumulation Assay

To allow drug uptake, cells were incubated in medium and amonafide (presence and absence of PKC412). Following uptake, the cells were washed with PBS and resuspended in fresh medium. An aliquot of cells from each sample was placed on ice for quantitation of baseline drug uptake. The remaining cells were incubated further and then resuspended in chilled fresh medium, and placed on ice for immediate flow cytometric analysis.

### Flow Cytometry

Functional expression analyses of Pgp were performed on a BD FACSCalibur flow cytometer (Franklin Lakes, N.J.). Cellular amonafide content was measured on the Cytopeia Influx flow cytometer (Seattle, Wash.). Data analysis was performed using the Dako Cytomation Summit software, version 4.0 (Fort Collins, Colo.). Cellular DiOC2 and amonafide content was assessed by analyzing cellular fluorescence of cells after efflux in the presence/absence of a Pgp inhibitor, either CSA or PKC412. The excitation and emission wavelengths used for DiOC2 and amonafide are 480/530 nm and 405/550 nm, respectively. Differential Pgp staining of anti-Pgp treated cells was compared to that of control cells counted with the same number of gated events. All test samples were compared to their respective controls using Kolmogorov-Smirnov (KS) non-parametric statistics and expressed as D-values ranging from 0 to 1. These analyses were performed using the NCSS 2007 software (Kaysville, Utah). This approach addresses the maximum difference between two empirical distribution functions.

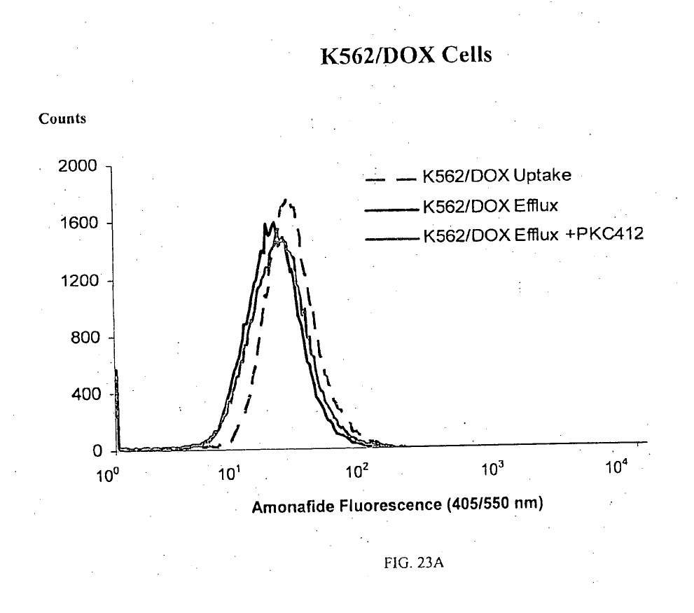

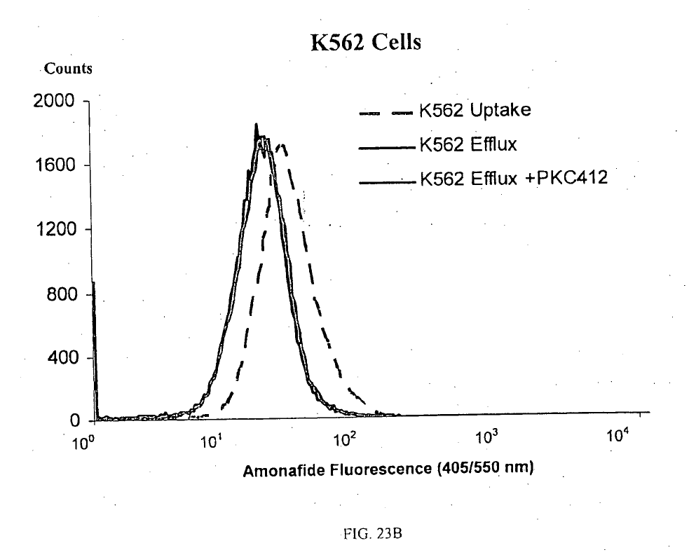

FIG. 22A and FIG. 22B show the results for DiOC2 in K562 and K562/Dox cells respectively. DiOC2 accumulated in Pgp negative K562 cells, but not in the Pgp positive K562/DOX cells. Addition of CSA (a Pgp inhibitor) reversed the Pgp-mediated efflux of DiOC2 in the K562/DOX cell lines and resulted in the measured KS D-value to be 88.9% of the K562 cells.

FIG. 23A and FIG. 23B show the results for amonafide in K562 and K562/Dox cells respectively. Amonafide uptake and efflux were not significantly different between the two cell lines and the amonafide content in each cell line did not significantly change in the presence of the Pgp inhibitor, PKC412, indicating that the amonafide cellular concentration is not affected by Pgp over-expression.

### Example 12

### Amonafide Retention in HL60NCR Cells

The uptake and efflux of amonafide was measured in the paired Pgp negative and Pgp positive cell lines, HL60-HL60/VCR.

### Drug Accumulation Assay

To allow drug uptake, cells were incubated in medium with increasing concentrations of amonafide or with a fixed concentration of amonafide (presence and absence of PSC833). Following uptake, the cells were washed with PBS and resuspended in fresh medium. An aliquot of cells from each sample was placed on ice for quantitation of baseline drug uptake. The remaining cells were incubated further and then resuspended in chilled fresh medium, and placed on ice for immediate flow cytometric analysis.

### Flow Cytometry

Functional expression analyses of Pgp were performed on a BD FACSCalibur flow cytometer (Franklin Lakes, N.J.). Cellular amonafide content was measured on the Cytopeia Influx flow cytometer (Seattle, Wash.). Data analysis was performed using the Dako Cytomation Summit software, version 4.0 (Fort Collins, Colo.). Amonafide content was assessed by analyzing cellular fluorescence of cells after efflux in the presence/absence of a Pgp inhibitor, PSC833. The excitation and emission wavelengths used for amonafide are 405/550 nm, respectively.

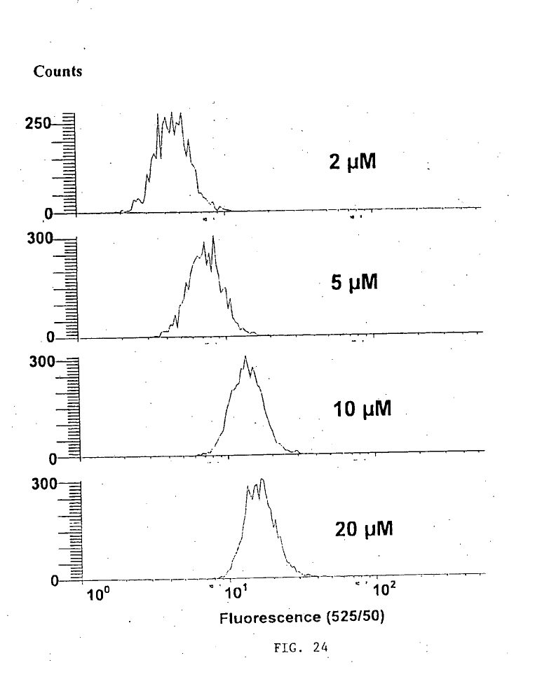

FIG. 24 shows that amonafide accumulation increases with increasing amonafide dose in the Pgp negative HL60 cell line.

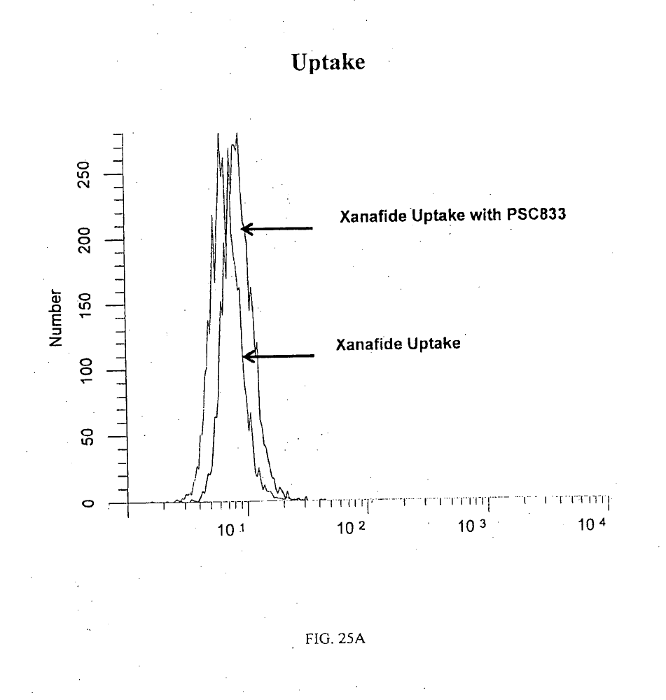

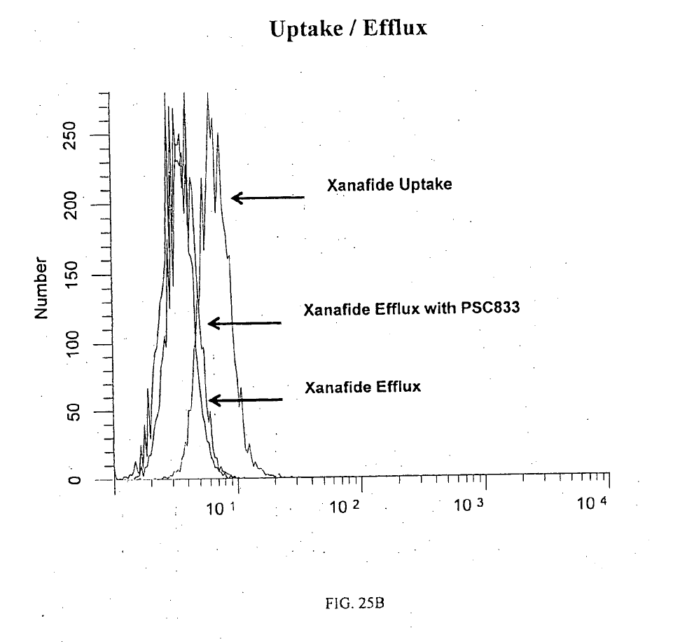

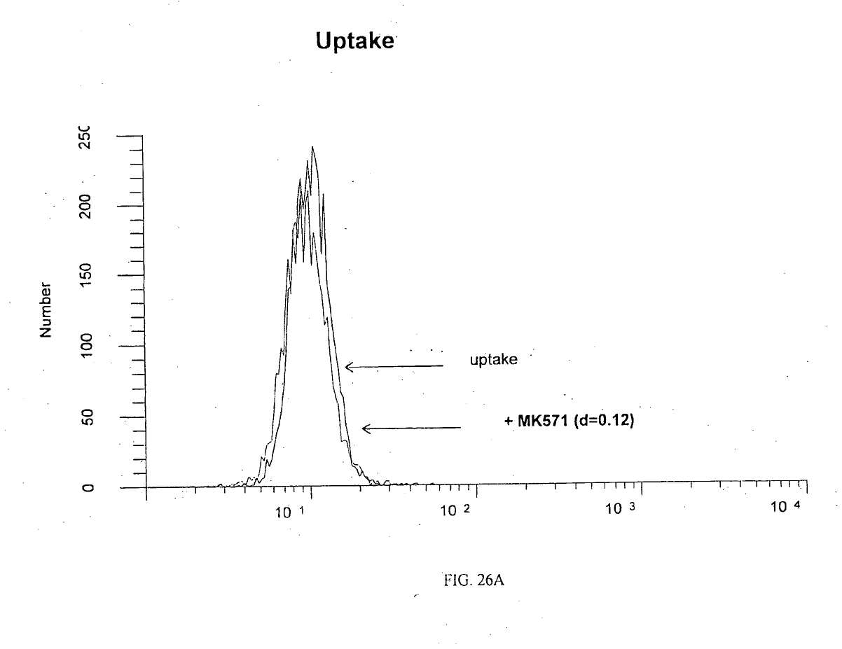

FIG. 25A and FIG. 25B show the results for amonafide HL60/VCR cells. Amonafide uptake and efflux did not significantly change in the presence of the Pgp inhibitor, PSC833, indicating that the amonafide cellular concentration is not affected by Pgp over-expression.

### Example 13

### Amonafide Retention in HL60/ADR Cells

The uptake and efflux of amonafide was measured in the MRP-1 positive cell lines, HL60/ADR.

### Drug Accumulation Assay

To allow drug uptake, cells (1×10⁶ cells/ml) were incubated in medium and amonafide (presence and absence of PKC412). Following uptake, the cells were washed with PBS and resuspended in fresh medium. An aliquot of cells from each sample was placed on ice for quantitation of baseline drug uptake. The remaining cells were incubated further and then resuspended in chilled fresh medium, and placed on ice for immediate flow cytometric analysis.

### Flow Cytometry

Functional expression analyses of MRP-1 were performed on a BD FACSCalibur flow cytometer (Franklin Lakes, N.J.). Cellular amonafide content was measured on the Cytopeia Influx flow cytometer (Seattle, Wash.). Data analysis was performed using the Dako Cytomation Summit software, version 4.0 (Fort Collins, Colo.). Amonafide content was assessed by analyzing cellular fluorescence of cells after efflux in the presence/absence of a MRP-1 inhibitor, MK571. The excitation and emission wavelengths used for amonafide are 405/550 nm, respectively.

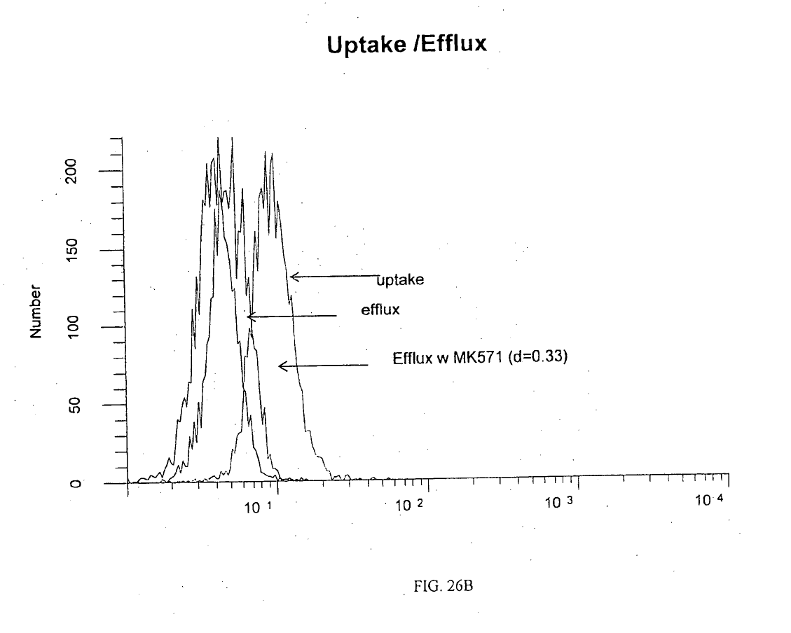

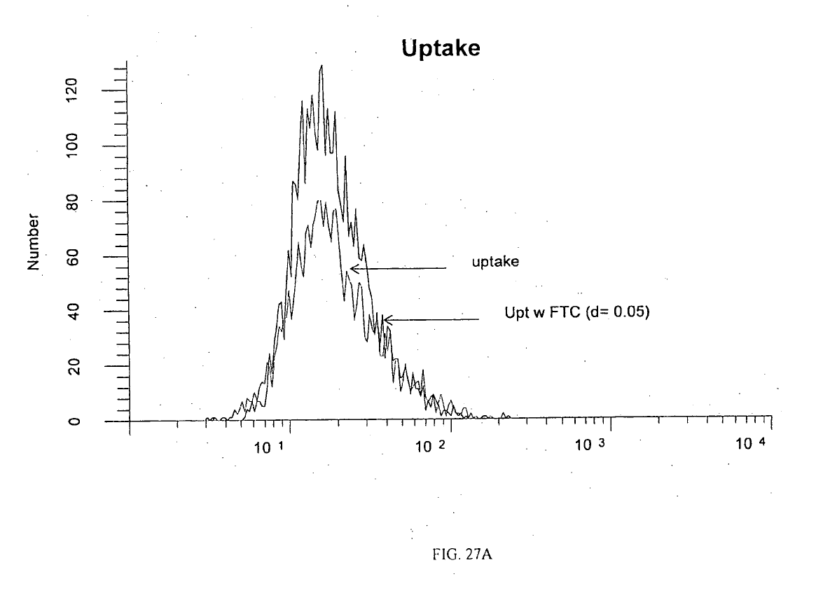

FIG. 26A and FIG. 26B show the results for amonafide HL60/VCR cells. Amonafide uptake and efflux did not significantly change in the presence of the MRP-1 inhibitor, MK571, indicating that the amonafide cellular concentration is not affected by MRP-1 over-expression.

### Example 14

### Amonafide Retention in 8226/MR20Cells

The uptake and efflux of amonafide was measured in the BCRP positive cell lines, 8226/MR20

### Drug Accumulation Assay

To allow drug uptake, cells (1×10⁶ cells/ml) were incubated in medium and amonafide (presence and absence of PKC412). Following uptake, the cells were washed with PBS and resuspended in fresh medium. An aliquot of cells from each sample was placed on ice for quantitation of baseline drug uptake. The remaining cells were incubated further and then resuspended in chilled fresh medium, and placed on ice for immediate flow cytometric analysis.

### Flow Cytometry

Functional expression analyses of BCRP were performed on a BD FACSCalibur flow cytometer (Franklin Lakes, N.J.). Cellular amonafide content was measured on the Cytopeia Influx flow cytometer (Seattle, Wash.). Data analysis was performed using the Dako Cytomation Summit software, version 4.0 (Fort Collins, Colo.). Amonafide content was assessed by analyzing cellular fluorescence of cells after efflux in the presence/absence of a BCRP inhibitor, FTC. The excitation and emission wavelengths used for amonafide are 405/550 nm, respectively.

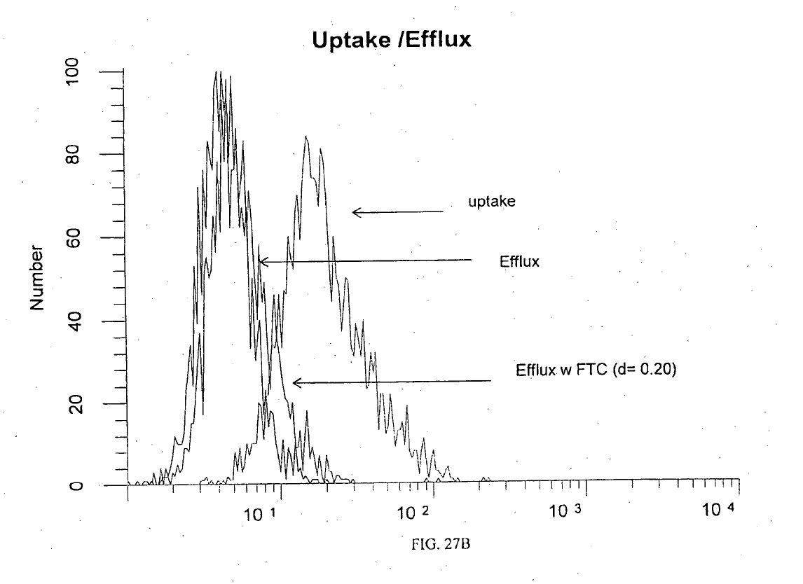

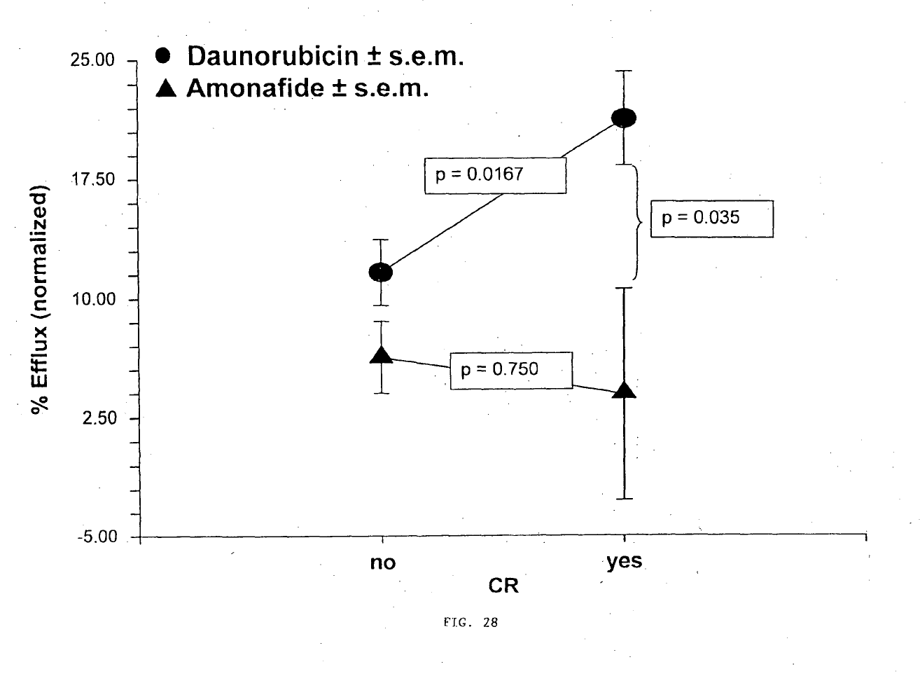

FIG. 27A and FIG. 27B show the results for amonafide in 8226/MR20 cells. Amonafide uptake and efflux did not significantly change in the presence of the BCRP inhibitor, FTC, indicating that the amonafide cellular concentration is not affected by BCRP over-expression.

### Example 15

### Drug Transport in Secondary AML Patient Cells

The expression and function of Pgp, MRP-1 and BCRP was measured in cells collected from patients with secondary AML. Pgp, MRP-1 and BCRP expression was measured by flow cytometry with the MRK16, MRPm6 and BXP21 antibodies, and function by modulation of uptake of the fluorescent substrates DiOC2(3), rhodamine-123 and pheophorbide A by PSC-833, MK571 and FTC, respectively, all measured by the Kolmogorov-Smirnov statistic, generating D-values.

Results are presented in Table 4. Pgp, MRP-1 and BCRP expression and/or function was observed in 18, 7 and 17 of 22 secondary AML samples, respectively. Cyclosporin A, which inhibits substrate drug efflux by Pgp, MRP-1 and BCRP, increased uptake of daunorubicin, idarubicin and amonafide L-malate by mean values of 19.7%, 7% and −2.5%, respectively, and increased uptake by ≧10% in 16, 12 and 5 patient samples. In conclusion, in relation to other topoisomerase 2 inhibitors used to treat AML, including daunorubicin, idarubicin, mitoxantrone, and etoposide, amonafide L-malate is a poor substrate for the MDR proteins expressed in AML cells in general, and S-AML cells in particular.

TABLE 4
Comparison of Drug transport for amonafide, daunorubicin and
idarubicin in secondary AML patient sample cells.

| | | | | | | | | | | CsA Modulation of Uptake | | |
|---|---|---|---|---|---|---|---|---|---|---|---|---|
| | | | | | | | | | | Amonafide | | |
| | Age/ | Prior | Cytotoxic | Pgp | | MRP-1 | | BCRP | | L- | | |
| Pt | Sex | MDS | therapy | Expr | Fxn | Expr | Fxn | Expr | Fxn | Malate | Daunorubicin | Idarubicin |
| 1 | 49F | — | Breast | 0.13 | 0.36 | 0 | 0.13 | 0.1 | 0 | 0 | 0.2 | 0 |
| 2 | 72M | CMML | — | 0.27 | 0.1 | 0.1 | 0.05 | 0.25 | 0.09 | 0.09 | 0.14 | 0.23 |
| 3 | 62F | MDS | — | 0.48 | 0.49 | 0.08 | 0.13 | 0.17 | 0.21 | 0 | 0.27 | 0.22 |
| 4 | 79M | MDS | — | 0.08 | 0.24 | 0.24 | 0.16 | 0.18 | 0.15 | 0.02 | 0.23 | 0.17 |
| 5 | 50F | — | Breast | 0.07 | 0 | 0 | 0 | 0.9 | 0.03 | 0 | 0 | 0 |
| 6 | 78M | MDS | — | 0.2 | 0 | 0.22 | 0 | 0.35 | 0 | 0.1 | 0.41 | 0.21 |
| 7 | 66M | MDS/ | Prostate | 0 | 0.35 | 0.54 | 0 | 0.88 | 0 | 0.31 | 0.47 | 0.21 |
| | | MPD | | | | | | | | | | |
| 8 | 79M | — | Bladder | 0.29 | 0.17 | 0.27 | 0 | 0.82 | 0 | 0.07 | 0.31 | 0.16 |
| 9 | 67M | MDS | — | 0.05 | 0.2 | 0.13 | 0.04 | 0.25 | 0.02 | 0 | 0.08 | 0.18 |
| 10 | 72M | MDS | — | 0.08 | 0.09 | 0.24 | 0 | 0.6 | 0 | 0.18 | 0.08 | 0.02 |
| 11 | 70M | MDS | — | 0.19 | 0 | 0.48 | 0 | 0.66 | 0 | 0 | 0.03 | 0.05 |
| 12 | 70F | MDS | — | 0.33 | 0.13 | 0.1 | 0 | 0.69 | 0 | 0 | 0.37 | 0.08 |
| 13 | 74M | MDS | — | 0 | 0.06 | 0 | 0 | 0 | 0.11 | 0.03 | 0.16 | 0.06 |
| 14 | 56F | — | NHL, | 0.64 | 0.11 | 0.16 | 0.02 | 0.21 | 0.22 | 0.05 | 0.29 | 0.28 |
| | | | Breast | | | | | | | | | |
| 15 | 75F | — | Breast | 0 | 0.24 | 0.18 | 0.14 | 0.38 | 0 | 0 | 0.07 | 0.16 |
| 16 | 77M | MDS | Lung | 0.87 | 0.12 | 0.13 | 0 | 0 | 0 | 0 | 0.08 | 0.03 |
| 17 | 62M | MDS | — | 0.34 | 0.02 | 0 | 0 | 0.51 | 0 | 0 | 0.24 | 0 |
| 18 | 76F | — | Breast | 0 | 0.12 | 0 | 0.05 | 0.69 | 0 | 0 | 0.03 | 0 |
| 19 | 76F | MDS | — | 0 | 0.15 | 0.11 | 0 | 0.45 | 0 | 0 | 0.04 | 0.17 |
| 20 | 72M | MDS | — | 0.26 | 0.21 | 0 | 0.03 | 0.24 | 0.03 | 0.15 | 0.35 | 0.23 |
| 21 | 65M | MDS | — | 0.51 | 0.12 | 0.11 | 0.1 | 0 | 0.11 | 0.43 | 0.12 | 0.19 |
| 22 | 87F | | CLL | 0.19 | 0.05 | 0 | 0.09 | 0.09 | 0.2 | 0 | 0.43 | 0.31 |

### Example 16

### Amonafide and Daunorubicin Efflux in Secondary AML Patients Treated with Amonafide+Cytarabine Combination Therapy

The efflux of amonafide and daunorubicin were measured in cryopreserved cells from 15 patients treated with amonafide+cytarabine. Cryopreserved cells were tested for viability and samples with viabilities less than 40% were considered inevaluable and discarded.

To measure drug uptake, the substrates were incubated with cells in medium containing each drug alone, or in combination with the modulator at the desired final concentrations. Cells were then washed and resuspended in PBS, and placed on ice. Drug-associated fluorescence was measured by flow cytometry using a FacScan flow cytometer (Becton Dickinson Immunocytometry Systems, San Jose, Calif.) equipped in standard fashion with an Argon laser for 488 nm excitation and a 585/42 band-pass filter (FL2) or a 670 long-pass (FL3) filter for emission collection. Data were analyzed with WinList software (Verity Software House, Topsham, Me.).

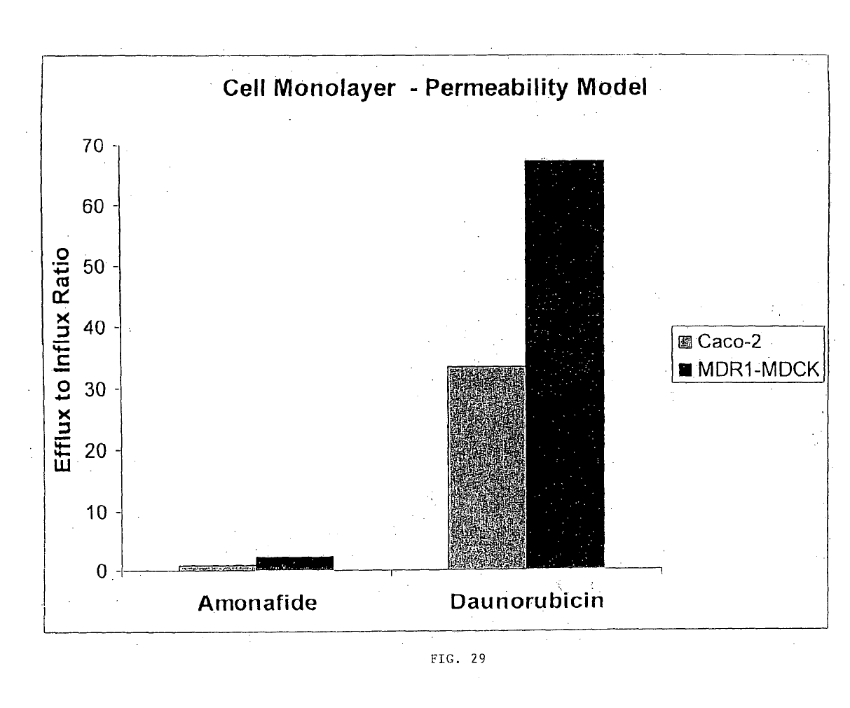

FIG. 28. shows that the efflux of Daunorubicin was negatively correlated with response, i.e non-complete responders had significantly higher efflux of daunorubicin then those patients who achieved a complete response (CR). In contrast there was no significant difference in amonafide efflux between patients achieving CR and those who did not.

### Example 17

### Amonafide Efflux in Cell Monolayer—Permeability Models

The procedure described below was adopted from Artursson P, et al., Caco-2 monolayers in experimental and theoretical predictions of drug transport. Adv Drug Deliv Rev. 2001 Mar. 1; 46(1-3):27-43, the relevant teachings of which is hereby incorporated by reference.

Caco-2 cells adopt colonic cell morphology and express many intestinal transport proteins and other enzymes when cultured under proper conditions. They also form tight junctions with each other. These limit the paracellular permeability or the “leakiness” of cell monolayers grown to confluence on polycarbonate membrane filters. This property makes Caco-2 monolayers a good test system for discriminating between passive absorption via the transcellular route and diffusion between cells via the paracellular route. MDR1-MDCK are Madin Darby Canine Kidney cells transfected with the human multi-drug resistance gene. Confluent monolayers made from these cells can be used to access a test compound's potential role as a P-gp substrate. The assay set up is similar to the CaCo-2 assay. Daunorubicin a known P-gp substrate was used as a control.

Caco-2 cell permeability studies were performed using Caco-2 cell monolayers grown on microporous membranes in multiwell insert systems. With the inserts suspended in the wells of multiwell plates, test compounds (5 μM) were added to either the upper (apical) or lower (basolateral) chamber to measure permeability in the absorptive (apical to basolateral) or secretive (basolateral to apical) directions, respectively. Samples were then taken from the opposite chamber at 120 minutes to measure the amount of test compound that has crossed the cell monolayer. The samples were analyzed using LC/MS detection. The parameter that is calculated from this data is the apparent permeability (Pₐₚₚ). This is the slope of the basolateral concentration versus time curve divided by the concentration of compound in the apical dosing chamber and the total area of the Caco-2 cell monolayer. Daunorubicin, a known P-gp substrate, was used as a control.

A compound is classified as having high efflux if the ratio of Pₐₚₚ(B-A)/Pₐₚₚ(A-B) is ≧3.0 and if the Pₐₚₚ(B-A) is ≧1.0×10⁻⁶ cm/s. Amonafide has a ratio of Pₐₚₚ(B-A)/Pₐₚₚ(A-B) of 1 and a Pₐₚₚ(B-A) of 26.8×10⁻⁶ cm/s. Therefore, as both criteria are not met, Amonafide is classified as not having significant efflux and as such is not a substrate for P-gp.

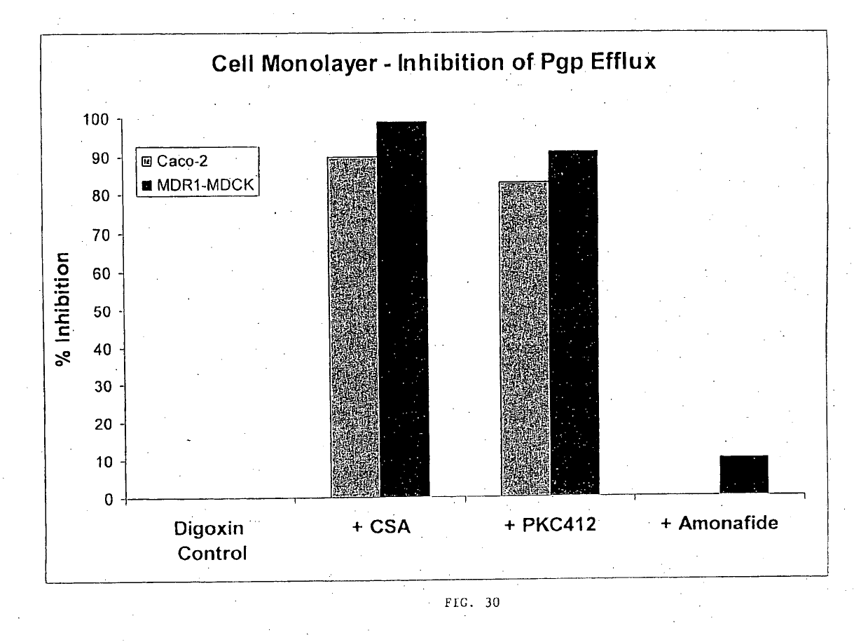

As is shown in Table 5A and FIG. 29, for both the Caco-2 (colon) and MDR1-MDCK (kidney) cell lines, the efflux/influx ratios for daunorubicin were high, 33.4 and 67.4, respectively. In both cell lines, efflux was effectively abrogated following addition of CSA, a known inhibitor of Pgp-mediated efflux. Amonafide, however, maintained similar efflux/influx ratios of 0.9 and 2.2 in both cell lines; addition of CSA did not have any effect on transmembrane amonafide flux.

The alternative possibility that amonafide might have served as a Pgp inhibitor in the previous experiment was also tested; these data are presented in Table 5B and FIG. 30. Both the Caco-2 and MDR1-MDCK cells were used for this model. Bidirectional digoxin flux was measured in the absence or presence of CSA, PKC412, or amonafide. For the Caco-2 cells, CSA and PKC412 resulted in 90% and 83% inhibition of digoxin efflux, respectively. Amonafide had no effect on digoxin efflux. In the MDR1-MDCK, transfected Pgp over-expressing cell line, CSA and PKC412 inhibited digoxin efflux by 99% and 91%, respectively. Amonafide caused a slight inhibitory effect of 10%.

TABLES 5A AND 5B
Calculated efflux/influx ratios for the indicated
test drugs in the Caco-2 and MDR1-MDCK cells.
A

| | Caco-2 | | MDR1-MDCK | |
|---|---|---|---|---|
| DRUG | Drug alone | +CSA | Drug alone | +CSA |
| Amonafide | 0.9 ± 0.2 | 1.1 ± 0.4 | 2.2 ± 0.8 | 1.2 ± 0.8 |
| Daunorubicin | 33.4 ± 8.1 | 1.0 ± 0.3 | 67.4 ± 18.6 | 1.0 ± 0.7 |

B

| | Caco-2 | | MDR1-MDCK | |
|---|---|---|---|---|
| | | % Inhib. | | % Inhib. |
| Test Compound | Efflux | of control | Efflux | of control |
| Digoxin | 10.6 ± 1.4 | NA | 70 + 11 | NA |
| Control | | | | |
| Digoxin + | 1.0 ± 0.04 | 90% | 0.9 + 0.2 | 99% |
| CSA | | | | |
| Digoxin + | 1.8 ± 1.3 | 83% | 6.4 + 0.8 | 91% |
| PKC412 | | | | |
| Digoxin + | 11.9 ± 0.5 | 0% | 63.2 + 18.6 | 10% |
| Amonafide | | | | |

These studies support the conclusion that amonafide is neither a substrate nor an inhibitor of Pgp.

### Example 18

### Correlation of Resistance Protein Expression and Activity of Amonafide in the Sixty Cell Lines of the NCI Oncology Screening Panel

The NCI oncology screening panel uses 60 cell lines representing a variety of types of cancer (see Table 6).

TABLE 6
The 60 cell lines in the NCI Oncology Screening panel

| Cell Line Name | Cancer Subtype |
|---|---|
| CCRF-CEM | Leukemia |
| HL-60(TB) | Leukemia |
| K-562 | Leukemia |
| MOLT-4 | Leukemia |
| RPMI-8226 | Leukemia |
| SR | Leukemia |
| A549/ATCC | Non-Small Cell Lung |
| EKVX | Non-Small Cell Lung |
| HOP-62 | Non-Small Cell Lung |
| HOP-92 | Non-Small Cell Lung |
| NCI-H226 | Non-Small Cell Lung |
| NCI-H23 | Non-Small Cell Lung |
| NCI-H322M | Non-Small Cell Lung |
| NCI-H460 | Non-Small Cell Lung |
| NCI-H522 | Non-Small Cell Lung |
| COLO 205 | Colon |
| HCC-2998 | Colon |
| HCT-116 | Colon |
| HCT-15 | Colon |
| HT29 | Colon |
| KM12 | Colon |
| SW-620 | Colon |
| SF-268 | CNS |
| SF-295 | CNS |
| SF-539 | CNS |
| SNB-19 | CNS |
| SNB-75 | CNS |
| U251 | CNS |
| LOX IMVI | Melanoma |
| MALME-3M | Melanoma |
| M14 | Melanoma |
| SK-MEL-2 | Melanoma |
| SK-MEL-28 | Melanoma |
| SK-MEL-5 | Melanoma |
| UACC-257 | Melanoma |
| UACC-62 | Melanoma |
| IGR-OV1 | Ovarian |
| OVCAR-3 | Ovarian |
| OVCAR-4 | Ovarian |
| OVCAR-5 | Ovarian |
| OVCAR-8 | Ovarian |
| SK-OV-3 | Ovarian |
| 786-0 | Renal |
| A498 | Renal |
| ACHN | Renal |
| CAKI-1 | Renal |
| RXF 393 | Renal |
| SN12C | Renal |
| TK-10 | Renal |
| UO-31 | Renal |
| PC-3 | Prostate |
| DU-145 | Prostate |
| MCF7 | Breast |
| NCI/ADR-RES | Breast |
| MDA-MB-231/ATCC | Breast |
| HS 578T | Breast |
| MDA-MB-435 | Breast |
| MDA-N | Breast |
| BT-549 | Breast |
| T-47D | Breast |

The cytotoxic activity of a vast number of anticancer agents have been determined in these lines. In addition, the differential gene expression profiles of these cell lines have been established. The data are publicly available and were obtained from the following websites:
http://genome-www.stanford.edu/nci60/
http://discover.nci.nih.gov/datasetsNature2000.jsp
http://www.broad.mit.edu/tools/data.html

The data was mined to calculate “Pearson Coefficients” for a series of agents including Amonafide. A Pearson Coefficient correlates drug activity to gene expression. In other words if the drug retains activity in cell lines expressing higher levels of a specific gene then the Pearson coefficient will be positive, if the drug loses activity in cells expressing high levels of the gene then the Pearson coefficient will be negative. If the level of gene expression has no impact on the activity of the drug then the Pearson Coefficient will be 0.

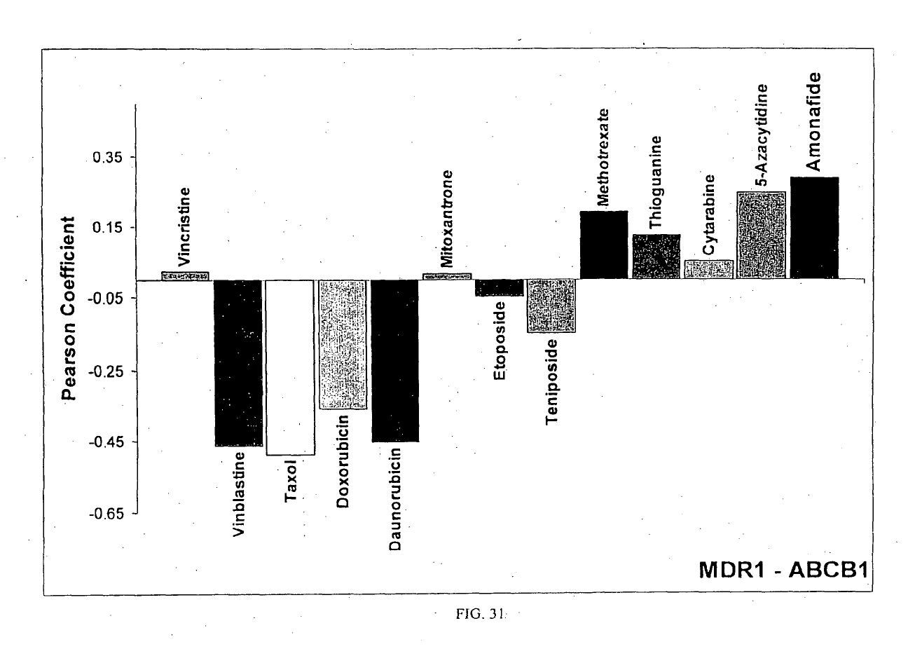

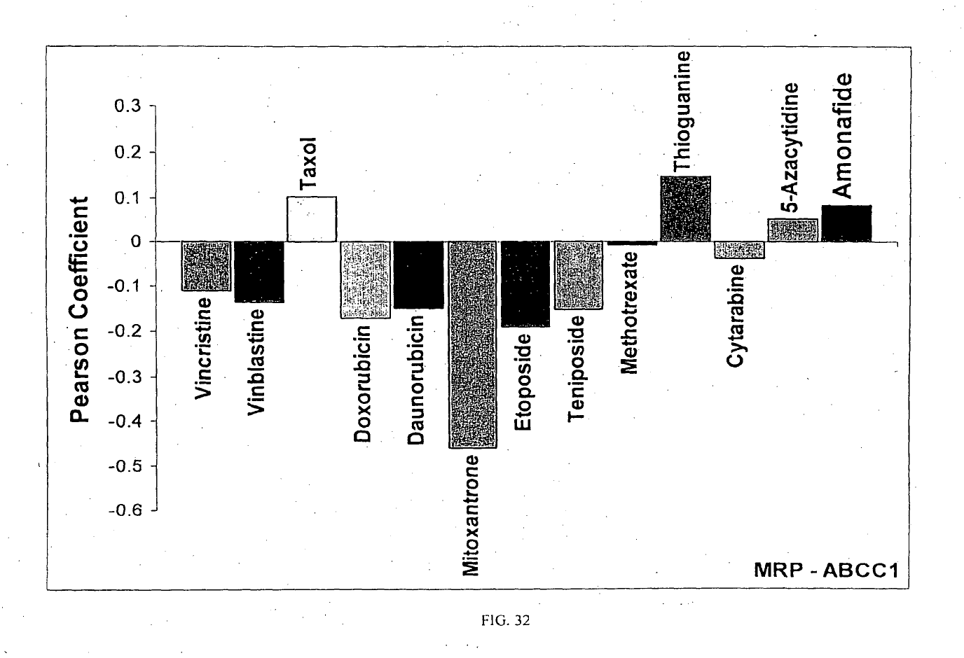

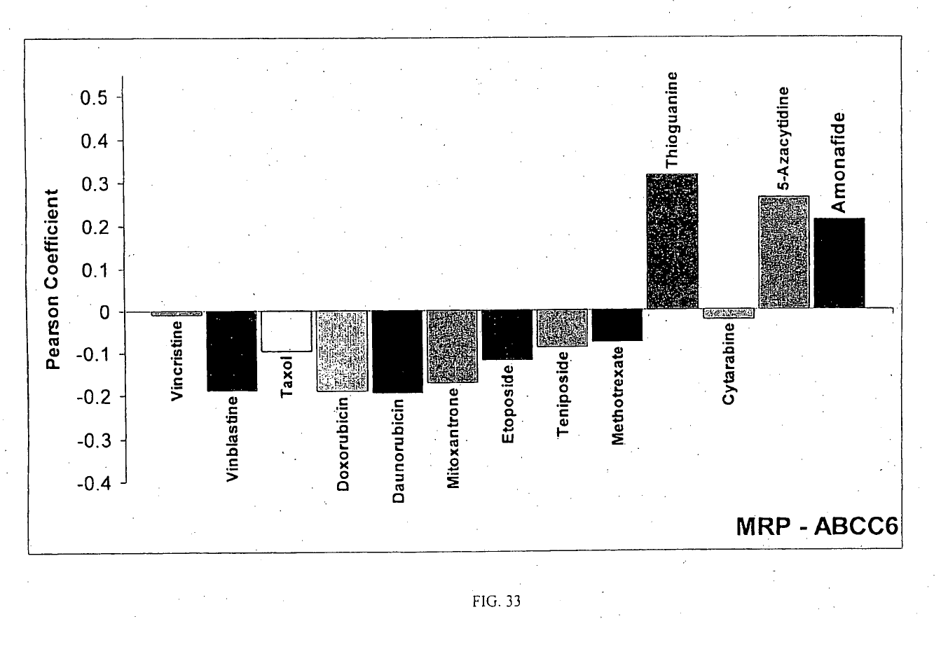

The Pearson coefficients were calculated for 13 drugs and 3 drug transporter genes associated with multidrug resistance ABCB1, ABCC1 and ABCC6. The results are presented in FIG. 31, FIG. 32 and FIG. 33 respectively.

Amonafide has a positive Pearson coefficient for all three drug transporter genes indicating that it retains its activity in cell lines expressing increased levels of these genes. In contrast, classical topoisomerase II inhibitors doxorubicin and daunorubicin have negative Pearson coefficients. These agents are known substrates for the ABCB1 gene product, p-glycoprotein (P-gp).

While this invention has been particularly shown and described with references to example embodiments thereof, it will be understood by those skilled in the art that various changes in form and details may be made therein without departing from the scope of the invention encompassed by the appended claims.

1. A method of treating a multidrug resistant cancer leukemia in a patient, wherein the multidrug resistance is mediated by expression of a p-glycoprotein ATP-binding cassette transporter (PUP), said method comprising administering to said patient a therapeutically effective amount of a compound of formula (I) or a pharmaceutically acceptable salt thereof: 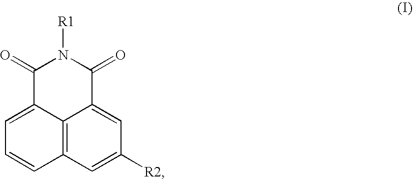
wherein
R1 is —(CH₂)ₙNR3R4;
R2 is —ORS, halogen, —NR6R7, —NHR6R7, sulphonic acid, nitro, —NR5COOR5, —NR5COR5 or —OCOR5;
R3 and R4 are independently H, C1-C4 alkyl group or, taken together with the nitrogen atom to which they are bonded, a non-aromatic nitrogen-containing heterocyclic group;
each R5 is independently H or a C1-C4 alkyl group;
R6 and R7 are independently H, a C1-C4 alkyl group or, taken together with the nitrogen atom to which they are bonded, a non-aromatic nitrogen-containing heterocyclic group; and
n is an integer from 0-3.

2. The method of claim 1, wherein the compound is represented by formula (II): 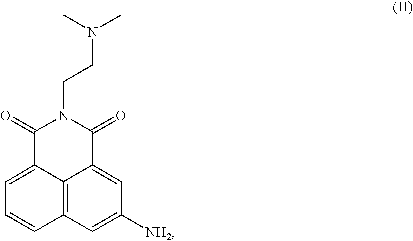
or a pharmaceutically acceptable salt thereof.

3. The method of claim 1, wherein the compound of formula (I) is a malate salt.

4-6. (canceled)

7. The method of claim 1, wherein the cancer is resistant to one or more anthracyclines.

8. The method of claim 7, wherein the anthracycline is daunorubicin or doxorubicin.

9. The method of claim 1, wherein the cancer is resistant to one or more vinca alkaloids.

10. The method of claim 9, wherein the vinca alkaloid is vinblastine or vincristine.

11. (canceled)

12. The method of claim 18, wherein the AML is a refractory leukemia.

13. The method of claim 12, wherein the compound of formula (I) is represented by formula (II): 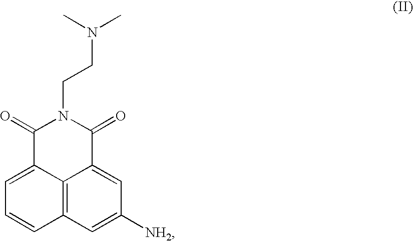
or a pharmaceutically acceptable salt thereof.

14. The method of claim 12, wherein the compound of formula (I) is a malate salt.

15. The method of claim 18, wherein the AML is a relapsed leukemia.

16. The method of claim 15, wherein the compound of formula (I) is represented by formula (II): 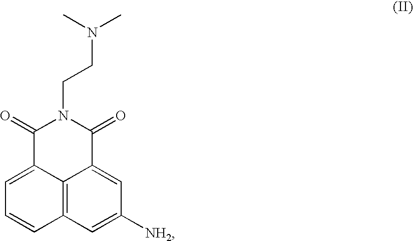
or a pharmaceutically acceptable salt thereof.

17. The method of claim 15, wherein the compound of formula (I) is a malate salt.

18. The method of claim 1 wherein the multidrug resistant leukemia is multidrug resistant acute myelogneous leukemia (AML).
